SEQUENCE LISTING (41 sequences)

SEQ ID NO: 1 - protein, 7 aa, synthetic construct
SNYLYDN

SEQ ID NO: 2 - protein, 9 aa, synthetic construct
REFPFYGDY

SEQ ID NO: 3 - protein, 6 aa, synthetic construct
EKHHSH

SEQ ID NO: 4 - protein, 38 aa, synthetic construct
DSHEKRHHGYRRKFHEKHHSHREFPFYGDYGSNYLYDN

SEQ ID NO: 5 - protein, 27 aa, synthetic construct
RKFHEKHHSHREFPFYGDYGSNYLYDN

SEQ ID NO: 6 - protein, 32 aa, synthetic construct
DSHAKRHHGYKRKFHEKHHSHRGYRSNYLYDN

SEQ ID NO: 7 - protein, 15 aa, synthetic construct
EKHHSHREFPFYGDY

SEQ ID NO: 8 - protein, 17 aa, synthetic construct
REFPFYGDYGSNYLYDN

SEQ ID NO: 9 - protein, 19 aa, synthetic construct
SHREFPFYGDYGSNYLYDN

SEQ ID NO: 10 - protein, 21 aa, synthetic construct
HHSHREFPFYGDYGSNYLYDN

SEQ ID NO: 11 - protein, 23 aa, synthetic construct
EKHHSHREFPFYGDYGSNYLYDN

SEQ ID NO: 12 - protein, 25 aa, synthetic construct
FHEKHHSHREFPFYGDYGSNYLYDN

SEQ ID NO: 13 - protein, 19 aa, synthetic construct
RKFHEKHHSHREFPFYGDY

SEQ ID NO: 14 - protein, 15 aa, synthetic construct
EKHHSHREFPFYGDY

SEQ ID NO: 15 - protein, 21 aa, synthetic construct
RKFHEKHHSHREFPFYGDYGS

SEQ ID NO: 16 - protein, 17 aa, synthetic construct
EKHHSHREFPFYGDYGS

SEQ ID NO: 17 - protein, 23 aa, synthetic construct
RKFHEKHHSHREFPFYGDYGSNY

SEQ ID NO: 18 - protein, 19 aa, synthetic construct
EKHHSHREFPFYGDYGSNY

SEQ ID NO: 19 - protein, 25 aa, synthetic construct
RKFHEKHHSHREFPFYGDYGSNYLY

SEQ ID NO: 20 - protein, 21 aa, synthetic construct
EKHHSHREFPFYGDYGSNYLY

SEQ ID NO: 21 - protein, 17 aa, synthetic construct
RKFHEKHHSHREFPFYG

SEQ ID NO: 22 - protein, 13 aa, synthetic construct
EKHHSHREFPFYG

SEQ ID NO: 23 - protein, 19 aa, synthetic construct
HHSHREFPFYGDYGSNYLY

SEQ ID NO: 24 - protein, 17 aa, synthetic construct
SHREFPFYGDYGSNYLY

SEQ ID NO: 25 - protein, 15 aa, synthetic construct
SHREFPFYGDYGSNY

SEQ ID NO: 26 - protein, 15 aa, synthetic construct
FPFYGDYGSNYLYDN

SEQ ID NO: 27 - protein, 13 aa, synthetic construct
FYGDYGSNYLYDN

SEQ ID NO: 28 - protein, 15 aa, synthetic construct
HHSHREFPFYGDYGS

SEQ ID NO: 29 - protein, 13 aa, synthetic construct
SHREFPFYGDYGS

SEQ ID NO: 30 - protein, 24 aa, synthetic construct
DSHAKRHHGYKRKFHEKHHSHRGY

SEQ ID NO: 31 - protein, 14 aa, synthetic construct
GYKRKFHEKHHSHR

SEQ ID NO: 32 - protein, 11 aa, synthetic construct
SHREFPFYGDY

SEQ ID NO: 33 - protein, 44 aa, synthetic construct
GGDSHEKRHHGYRRKFHEKHHSHREFPFYGDYGSNYLYDNLPET

SEQ ID NO: 34 - protein, 11 aa, synthetic construct
VGDGGLFEKKL

SEQ ID NO: 35 - protein, 5 aa, synthetic construct
  VARIANT 3: note = Wherein X is R, K, H, D or E
SHXGY

SEQ ID NO: 36 - protein, 4 aa, synthetic construct
DGGL

SEQ ID NO: 37 - protein, 12 aa, synthetic construct
HHIYLGAVNYIY

SEQ ID NO: 38 - protein, 5 aa, synthetic construct
  MOD_RES 5: note = Adamantylated Gly
DGGLG

SEQ ID NO: 39 - protein, 12 aa, synthetic construct
  REGION 12: note = N-terminus (NH2)
HHIYLGAVNYIY

SEQ ID NO: 40 - protein, 14 aa, synthetic construct
KRLFKEFLFSLRKY

SEQ ID NO: 41 - protein, 12 aa, synthetic construct
HHIYLGATNYIY

(19) United States
(12) Patent Application Publication
(10) Pub. No.: US 2025/0276038 A1
(43) Pub. Date: Sep. 4, 2025

(54) HISTATIN COMBINATIONS AND METHODS FOR TREATING OR INHIBITING CELL LOSS

(71) Applicant: VISUS THERAPEUTICS, INC., Seattle, WA (US)

(72) Inventors: James Albert BURKE, Santa Ana, CA (US); Rhett Mead SCHIFFMAN, Laguna Beach, CA (US)

(21) Appl. No.: 18/290,965

(22) PCT Filed: Jul. 22, 2022
(86) PCT No.: PCT/US2022/038031

§ 371 Date: Aug. 26, 2024

Related U.S. Application Data
(60) Provisional application No. 63/224,982, filed on Jul. 23, 2021.
(60) Provisional application No. 63/238,061, filed on Aug. 27, 2021.
(60) Provisional application No. 63/252,468, filed on Oct. 5, 2021.

(51) Int. Cl. A61K 38/18; A01N 1/122; A61K 38/17; A61K 45/06; A61P 27/02

(52) U.S. Cl. CPC A61K 38/185; A01N 1/122; A61K 38/1709; A61K 45/06; A61P 27/02

(57) ABSTRACT

The present disclosure provides methods and compositions for reducing or preventing loss of keratocytes, keratinocytes, and/or keratoblasts, restoring keratocytes, keratinocytes, and/or keratoblasts, inhibiting apoptosis to prevent loss of cells in the eye or skin caused by surgical procedure, injury, or disease, preventing or minimizing refractive regression and subepithelial scarring, treating corneal wounds, treating dermal or ophthalmic diseases, disorders and/or conditions, or storing a biological sample.

### FIELD OF THE INVENTION

The present disclosure relates to histatins and analogs thereof, optionally in combination with ciliary neurotrophic factor (CNTF) peptides and analogs thereof, for reducing or preventing loss of keratocytes, keratinocytes, and/or keratoblasts, restoring keratocytes, keratinocytes, and/or keratoblasts, inhibiting apoptosis to prevent loss of cells in the eye or skin caused by surgical procedure, injury or disease, preventing or minimizing refractive regression and subepithelial scarring, treating corneal wounds, treating dermal or ophthalmic diseases, disorders, and/or conditions, or storing a biological sample.

### BACKGROUND OF THE INVENTION

Histatins are naturally occurring oral and lacrimal peptides produced by humans and non-human primates that demonstrate direct anti-infective activity, have potent anti-inflammatory properties, and stimulate epithelial wound healing in several tissue and organ culture systems. Techniques have been developed to isolate this natural substance, making it an available topical treatment for wounds.

Ciliary neurotrophic factor (CNTF) is a cytokine of the interleukin-6 family that is sequestered in adult glia and whose production increases during injury. It is essential in the maintenance of dopaminergic neurons of the substantia nigra and spinal cord motor neurons; when administered to mice with neuromuscular degeneration, it produces neuroprotective effects.

Anti-apoptotic agents are compounds or peptides that inhibit cell death.

Injury to the cornea from a scrape, from photorefractive or surgical procedures, or from disease such as keratoconus can result in a loss of stromal keratocytes. It is thought that cell loss in this regard is driven by a process of apoptosis. A need exists to develop therapies to prevent or reduce loss of keratocytes, keratinocytes, and/or keratoblasts after surgery, injury, or disease.

### BRIEF SUMMARY OF THE INVENTION

The present disclosure provides methods of reducing or preventing loss of keratocytes, keratinocytes, and/or keratoblasts, restoring keratocytes, keratinocytes, and/or keratoblasts, inhibiting apoptosis to prevent loss of cells in the eye or skin caused by injury or disease, preventing or minimizing refractive regression and subepithelial scarring, mobilizing keratocytes, or treating corneal wounds, comprising administering to the eye or skin of a subject in need thereof a therapeutically effective amount of a composition comprising a histatin or analog thereof and/or a ciliary neurotrophic factor (CNTF) peptide or analog thereof, and, optionally, an anti-apoptotic agent.

In some aspects, the histatin is histatin-1 (SEQ ID NO:4).

In some aspects, the CNTF peptide is peptide 6 (SEQ ID NO: 34).

In some aspects, the composition comprises the histatin or analog thereof at a concentration of from about 0.1 μM to about 150 μM.

In some aspects, the composition comprises the histatin or analog thereof at a concentration of about 80 μM.

In some aspects, the composition comprises the CNTF peptide or analog thereof at a concentration of from about 0.1 μM to about 150 μM.

In some aspects, the composition comprises the CNTF peptide or analog thereof at a concentration of about 100 μM.

In some aspects, from about 1 μL to about 100 μL of the composition is administered to the eye or skin of the subject.

In some aspects, about 35 μL of the composition is administered to the eye or skin of the subject.

In some aspects, the composition is administered daily for from about 1 day to about 180 days.

In some aspects, the composition is administered daily for from about 1 to about 28 days.

In some aspects, the composition is administered one, two, three, or four times per day.

In some aspects, the composition is administered three times per day.

In some aspects, the composition is administered as an eye drop, gel, ointment, or tissue glue.

In some aspects, the composition is administered in an ocular insert or contact lens.

In some aspects, the subject is a mammal.

In some aspects, administering the composition results in a reduction in loss of keratocytes, keratinocytes, and/or keratoblasts.

In some aspects, loss of keratocytes, keratinocytes, and/or keratoblasts is caused by ocular infection, corneal injury, ocular surgery, ocular injury, ocular infection, ocular disease, or an ocular disorder.

In some aspects, the ocular surgery is glaucoma surgery, photorefractive keratectomy (PRK) surgery, or corneal refractive procedure.

In some aspects, the ocular disease is Fuch's corneal dystrophy.

In some aspects, the ocular disorder is keratoconus.

In some aspects, loss of keratocytes, keratinocytes, and/or keratoblasts is caused by skin photodamage.

In some aspects, loss of keratocytes, keratinocytes, and/or keratoblasts is caused by chemotherapy.

The present disclosure also provides methods of reducing or preventing loss of keratocytes, keratinocytes, and/or keratoblasts, restoring keratocytes, keratinocytes, and/or keratoblasts, inhibiting apoptosis to prevent loss of cells in the eye or skin caused by injury or disease, preventing or minimizing refractive regression and subepithelial scarring, mobilizing keratocytes, keratinocytes, and/or keratoblasts, or treating corneal wounds, comprising administering to the eye or skin of a subject in need thereof a therapeutically effective amount of a composition comprising a histatin or analog thereof, and a second active agent.

The present disclosure also provides methods of treating or prophylactically treating dermal or ophthalmic diseases, disorders, or conditions, comprising administering to the eye or skin of a subject in need thereof a therapeutically effective amount of a composition comprising a histatin or analog thereof and/or a ciliary neurotrophic factor (CNTF) peptide or analog thereof.

In some aspects, the composition further comprises an anti-apoptotic agent.

In some aspects, the dermal disease, disorder, or condition is post-CO₂ recovery, thermal burn, decubitus ulcer, autonomic dysreflexia in spinal cord injury, diabetic ischemic ulcer, ulcer prophylaxis, skin graft recovery, trauma resulting in skill loss, post-cryo/electrodesiccation recovery, or post-Mohs/skin cancer wound healing.

In some aspects, the ophthalmic disease, disorder, or condition is post-photorefractive keratectomy (PRK) surgery, glaucoma surgery, infectious corneal ulcer, dye eye disease, post-collagen crosslinking (CSX) for keratoconus, neurotrophic keratitis, Fuch's corneal dystrophy, contact lens intolerance, dry eye disease, keratoconus progression inhibition, posterior polymorphous corneal dystrophy (PPCD), aphakic or pseudophakic bullous keratopathy (ABK/PBK), endothelial dysfunction caused by penetrating or blunt trauma, congenital hereditary endothelial dystrophy (CHED), iridocorneal endothelial (ICE) syndrome, refractory glaucoma, previous failed corneal grafts, or herpes simplex virus endotheliitis.

In some aspects, wherein the ophthalmic disease, disorder, or condition is post-photorefractive keratectomy surgery, glaucoma surgery, or Fuch's corneal dystrophy.

In some aspects, the composition is administered by intraocular injection, subretinal injection, intravitreal injection, periocular administration, subconjuctival injection, suprachoroidal injections, retrobulbar injection, intracameral injection, or sub-Tenon's injection.

In some aspects, the composition is administered by topical administration.

The present disclosure also provides methods of storing a biological sample prior to a surgical procedure, comprising contacting the biological sample with a composition comprising a histatin or analog thereof and/or a CNTF peptide or analog thereof, and, optionally, an anti-apoptotic agent and storing said biological sample at a temperature of from about −10° C. to about 25° C.

In some aspects, the biological sample is donor tissue.

In some aspects, the donor tissue is corneal tissue.

In some aspects, the biological sample remains viable for about 0.5 days to about 90 days.

In some aspects, the surgical procedure is photorefractive keratectomy (PRK) surgery, Descemet stripping automated endothelial keratoplasty (DSAEK), or Descemet's membrane endothelial keratoplasty (DMEK).

The present disclosure also provides methods of reducing or preventing loss of corneal endothelial cells and/or restoring corneal endothelial cells comprising administering to the eye of a subject in need thereof a therapeutically effective amount of a composition comprising a histatin or analog thereof and, optionally, an anti-apoptotic agent.

In some aspects, the anti-apoptotic agent is a Fas inhibitor. In some aspects, the Fas inhibitor is MET12 (SEQ ID NO:37). In some aspects, the anti-apoptotic agent is a MET variant (SEQ ID NO:41).

In some aspects, administering the composition results in from about 20% to about 80% reduction in loss of corneal endothelial cells compared to no treatment.

In some aspects, the loss of corneal endothelial cells is caused by one or more of Fuch's endothelial corneal dystrophy, posterior polymorphous corneal dystrophy, aphakic or pseudophakic bullous keratopathy, endothelial dysfunction caused by penetrating or blunt trauma, congenital hereditary endothelial dystrophy, iridocorneal endothelial syndrome, refractory glaucoma, previous failed corneal grafts, surgical trauma, or herpes simplex virus endotheliitus.

In some aspects, the loss of corneal endothelial cells is caused by Fuch's endothelial corneal dystrophy.

In some aspects, the composition is administered prior to a surgical procedure.

In some aspects, the composition is administered during a surgical procedure.

In some aspects, the composition is administered after a surgical procedure.

In some aspects, the composition is administered both prior to and after a surgical procedure.

In some aspects, the composition is administered prior to, during and after the surgical procedure.

In some aspects, the surgical procedure is penetrating keratoplasty (PK), Descemet stripping automated endothelial keratoplasty (DSAEK), or Descemet's membrane endothelial keratoplasty (DMEK).

The present disclosure also provides methods of restoring, regenerating, or promoting regeneration of corneal nerves comprising administering to the eye of a subject in need thereof a therapeutically effective amount of a composition comprising a histatin or analog thereof, and/or a ciliary neurotrophic factor (CNTF) peptide or analog thereof, and, optionally, an anti-apoptotic agent.

In some aspects, administering the composition results in from about 20% to about 80% reduction in loss of and/or damage to corneal nerves compared to no treatment.

In some aspects, the loss of corneal nerves is caused by one or more of neurotrophic keratitis, dry eye disease, herpetic keratitis, leprosy, diabetes, keratoconjunctivitis sicca, or keratoconus.

In some aspects, the loss of or damage to corneal nerves is caused by a surgical procedure.

### BRIEF DESCRIPTION OF THE DRAWINGS

FIG. 1A is a line graph showing keratocyte counts in New Zealand white rabbits following surgical keratectomy after 0, 7, 10, 14, and 21 days while dosing with phosphate-buffered saline (PBS), histatin-1 (Hst-1), or a combination of histatin-1 and peptide 6 (P6) three times per day.

FIG. 1B is a line graph showing keratocyte counts in New Zealand white rabbits following surgical keratectomy after 0, 7, 10, 14, 21, and 28 days while dosing with phosphate-buffered saline (PBS), histatin-1 (Hst-1), or a combination of histatin-1 and peptide 6 (P6) three times per day.

FIG. 2 is a series of in vivo confocal microscopy images of rabbit corneas following surgical keratectomy after 0 (Baseline), 10, 14, and 21 days while dosing with phosphate-buffered saline (PBS) three times per day.

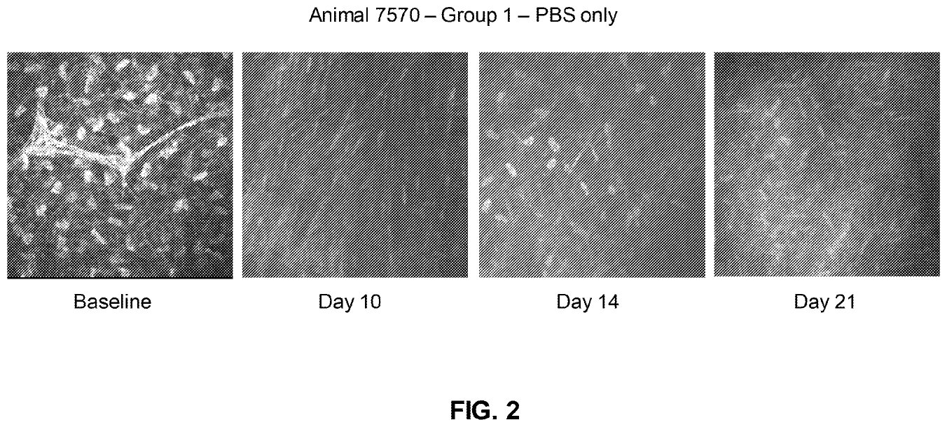

FIG. 3 is a series of in vivo confocal microscopy images of rabbit corneas following surgical keratectomy after 0 (Baseline), 10, 14, and 21 days while dosing with histatin-1 (Hst-1) three times per day.

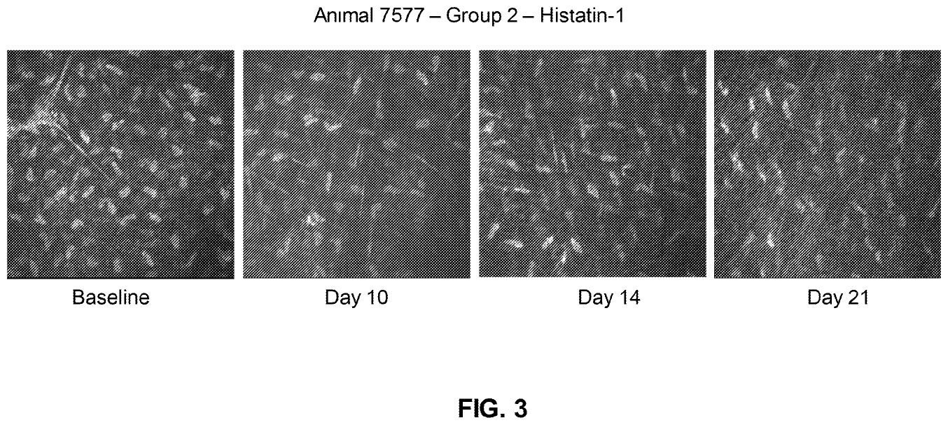

FIG. 4 is a series of in vivo confocal microscopy images of rabbit corneas following surgical keratectomy after 0 (Baseline), 10, 14, and 21 days while dosing with histatin-1 (Hst-1) and peptide 6 (P6) three times per day.

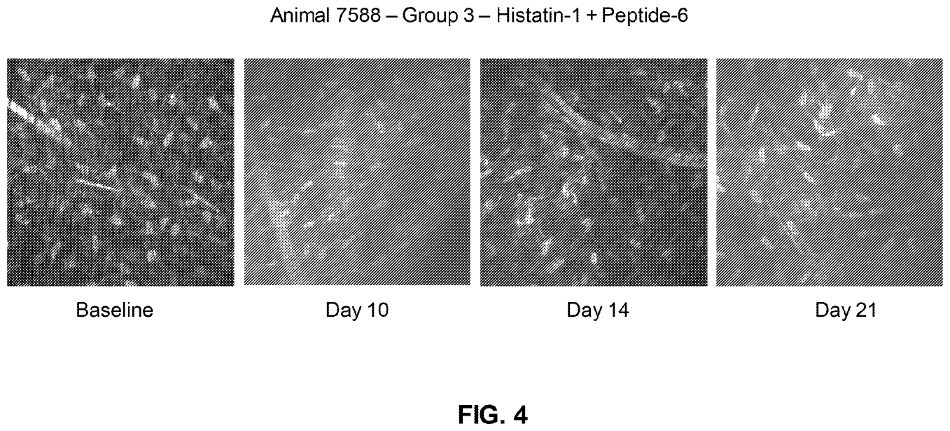

FIG. 5 is a line graph showing corneal endothelial cell density in New Zealand White rabbits following surgical anterior keratectomy after 0, 7, 10, 14, 21, and 28 days while dosing with topical phosphate-buffered saline (PBS) or histatin-1 (Hst-1) three times per day.

FIG. 6 is a bar graph showing corneal nerve regeneration in New Zealand white rabbits as measured by the percent of initial lesion area 28 days after surgical anterior keratectomy while dosing with phosphate-buffered saline (PBS), histatin-1 (Hst-1) or a combination of histatin-1 and peptide 6 (P6) three times per day.

FIG. 7 is a bar graph showing the percent area of corneal buttons stained with trypan blue (indicating tissue damage) between corneas stored in standard preservation solution and corneas stored in standard preservation solution plus Histatin-1.

### DETAILED DESCRIPTION OF THE INVENTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

“Percent identity” refers to the extent of identity between two sequences (e.g., amino acid sequences or nucleic acid sequences). Percent (%) identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” is art-recognized, and includes relatively non-toxic, inorganic and organic acid addition salts of compounds and relatively non-toxic, inorganic and organic base addition salts of compounds.

Inorganic acids that may be used to prepare pharmaceutically acceptable salts include, but are not limited to, hydrochloric acid, phosphoric acid, sulfuric acid, hydrobromic acid, hydroiodic acid, phosphorous acid and the like. Organic acids that may be used to prepare pharmaceutically acceptable salts include, without limitation, aliphatic mono-and dicarboxylic acids, such as tartaric acid, oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include, but are not limited to, hydrochloride, hydrobromide, nitrate, sulfate, pyrosulfate, bisulfate, sulfite, bisulfite, phosphate, monohydrogenphosphate, dihydrogenphosphate, metaphosphate, pyrophosphate, hydroiodide, hydrofluoride, acetate, propionate, formate, oxalate, tartrate, citrate, lactate, p-toluenesulfonate, methanesulfonate, and maleate. Suitable pharmaceutically acceptable salts may also be formed by reacting active agents with an organic base such as methylamine, ethylamine, ethanolamine, lysine, ornithine and the like. Pharmaceutically acceptable salts include the salts formed between carboxylate or sulfonate groups that may be found on some active agents and inorganic cations, such as sodium, potassium, ammonium, or calcium, or such organic cations as isopropylammonium, trimethylammonium, tetramethylammonium, and imidazolium. All of these salts may be prepared by conventional means from the active agents by reacting, for example, the appropriate acid or base with the active agents.

The term “excipient” refers to any substance, not itself a therapeutic agent, that may be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “unit dosage form” or “unit dose composition” as used herein refers to a device containing a quantity of the therapeutic compound, said quantity being such that one or more predetermined units may be provided as a single therapeutic administration.

The terms “administer,” “administering,” “administration,” and the like, as used herein, refer to methods that may be used to enable delivery of a drug, e.g., a histatin, to the desired site of biological action (e.g., to the eye or skin). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount of a therapeutic, e.g., a histatin, effective to treat a disease or disorder in a subject. In the case of disorders or injuries to the eye, the therapeutically effective amount of the therapeutic can mobilize keratocytes, prevent or restore corneal stromal keratocyte loss, prevent and restore loss of corneal stromal keratocytes caused by epithelial or stromal injury and disease, inhibit or prevent apoptosis to prevent and restore loss of cells in the eye and skin caused by injury or disease, and/or improve symptoms in patients with an ophthalmic condition or disorder, e.g., eye surgery or disease. A “prophylactically effective amount” refers to an amount effective to achieve the desired prophylactic result. A desired prophylactic result includes prevention of the full amount of damage to the eye (including corneal damage, nerve damage, cell death, etc.) expected to occur to a subject without administration of a composition of the invention. Effective prophylactic treatment does not mean that the subject to whom the composition is administered is free from any negative consequences of an eye disease or disorder, or damage post-surgical procedure, but (as compared to an untreated subject) will show some benefit in the amount of relatively healthy cells and/or nerves, and/or the treated subject has reduced symptoms as compared to an untreated subject. A prophylactically effective amount as applied to storage of biological samples prior to a surgical procedure means that the biological sample can be effectively stored for a longer time period prior to surgery than an untreated sample.

Terms such as “treating,” “treatment,” “to treat,” “alleviating,” and “to alleviate” refer to therapeutic measures that cure, slow down, lessen symptoms of, and/or halt progression of a pathologic condition or disorder. Thus, those in need of treatment include those already diagnosed with or suspected of having the disorder. In certain aspects, a subject is successfully “treated” for an ophthalmic and/or skin disease or disorder according to the methods of the present invention if the patient shows one or more of the following: mobilization of keratocytes, prevention or restoration of corneal stromal keratocyte loss, prevention and restoration of loss of corneal stromal keratocytes caused by epithelial or stromal injury and disease, inhibition or prevention of apoptosis to prevent, restoration of cells in the eye and skin, and/or improvement of one or more symptoms expressed by the subject prior to treatment.

Prophylactic or preventative measures refer to measures that prevent and/or slow the development of an ophthalmic and/or skin condition or disorder, or lessen the symptoms associated with ophthalmic damage, e.g., from eye surgery, injury, or disease. Thus, those in need of prophylactic or preventative measures include those prone to have the ophthalmic and/or skin condition or disorder and those in whom the ophthalmic and/or skin condition or disorder is to be prevented or those subjects who are to undergo eye surgery.

As used herein, “pretreatment or prophylaxis” means substantial absence of keratocyte, keratinocyte, and/or keratoblast loss, or reduced keratocyte, keratinocyte, and/or keratoblast loss by at least 20% and preferably 50% and more preferably 80% as measured over the course of 3 months, 6 months, 9 months, or one year.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited are not changed by the presence of more than that which is recited, but excludes prior art embodiments.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

### Description

The present disclosure provides methods of reducing or preventing loss of keratocytes, restoring keratocytes, inhibiting apoptosis to prevent loss of cells in the eye or skin caused by surgical procedure, injury or disease, preventing or minimizing refractive regression and subepithelial scarring, mobilizing keratocytes, or treating corneal wounds, comprising administering to a subject in need thereof a therapeutically effective amount of a composition comprising a (1) a histatin or analog thereof; (2) a histatin or analog thereof and a ciliary neurotrophic factor (CNTF) peptide or analog thereof; (3) a histatin or analog thereof and an anti-apoptotic agent; or (4) a histatin or analog thereof, a CNTF peptide or analog thereof, and an anti-apoptotic agent.

The present disclosure also provides methods of reducing or preventing loss of keratinocytes, restoring keratinocytes, inhibiting apoptosis to prevent loss of cells in the eye or skin caused by surgical procedure, injury or disease, preventing or minimizing refractive regression and subepithelial scarring, mobilizing keratinocytes, or treating corneal wounds, comprising administering to a subject in need thereof a therapeutically effective amount of a composition comprising a (1) histatin or analog thereof; (2) a histatin or analog thereof and a ciliary neurotrophic factor (CNTF) peptide or analog thereof; (3) a histatin or analog thereof and an anti-apoptotic agent; or (4) a histatin or analog thereof, a CNTF peptide or analog thereof, and an anti-apoptotic agent.

The present disclosure provides methods of reducing or preventing loss of keratoblasts, restoring keratoblasts, inhibiting apoptosis to prevent loss of cells in the eye or skin caused by surgical procedure, injury or disease, preventing or minimizing refractive regression and subepithelial scarring, mobilizing keratoblasts, or treating corneal wounds, comprising administering to a subject in need thereof a therapeutically effective amount of a composition comprising a (1) histatin or analog thereof; (2) a histatin or analog thereof and a ciliary neurotrophic factor (CNTF) peptide or analog thereof; (3) a histatin or analog thereof and an anti-apoptotic agent; or (4) a histatin or analog thereof, a CNTF peptide or analog thereof, and an anti-apoptotic agent.

The present disclosure also provides methods of treating dermal or ophthalmic diseases, disorders, and/or conditions comprising administering to a subject in need thereof a therapeutically effective amount of a composition comprising a (1) histatin or analog thereof; (2) a histatin or analog thereof and a ciliary neurotrophic factor (CNTF) peptide or analog thereof; (3) a histatin or analog thereof and an anti-apoptotic agent; or (4) a histatin or analog thereof, a CNTF peptide or analog thereof, and an anti-apoptotic agent.

The present disclosure also provides methods of storing a biological sample prior to a surgical procedure comprising contacting the biological sample with a composition comprising a (1) histatin or analog thereof; (2) a histatin or analog thereof and a ciliary neurotrophic factor (CNTF) peptide or analog thereof; (3) a histatin or analog thereof and an anti-apoptotic agent; or (4) a histatin or analog thereof, a CNTF peptide or analog thereof, and an anti-apoptotic agent.

Without wishing to be bound by any particular theory, the histatin or analog thereof and the CNTF peptide or analog thereof, and, optionally, the anti-apoptotic agent, prevent loss of keratocytes, keratinocytes, and/or keratoblasts in a subject through an anti-apoptosis mechanism. Other mechanisms that may explain this effect include the known effect of histatin-1 and the CNTF peptide or analog thereof, and, optionally, the anti-apoptotic agent as an inhibitor of inflammatory factors such as matrix metalloproteinases, which break down extracellular material. The histatin or analog thereof and the CNTF peptide or analog thereof, and, optionally, the anti-apoptotic agent provide a supportive environment in the cornea following injury. The histatin or analog thereof and the CNTF peptide or analog thereof, and, optionally, the anti-apoptotic agent can also promote new keratocyte generation from limbal stem cells. The CNTF peptide or analog thereof, and, optionally, the anti-apoptotic agent promote keratocyte restoration.

In some aspects, the histatin or analog thereof is histatin-1, which is represented by SEQ ID NO: 4 in Table 1 below. In some aspects, the histatin or analog thereof is histatin-2, which is represented by SEQ ID NO: 5 in Table 1 below. In some aspects, the histatin or analog thereof is histatin-3, which is represented by SEQ ID NO: 6 in Table 1 below. In some aspects, the histatin or analog thereof is histatin-5, which is represented by SEQ ID NO: 30 in Table 1 below. In some aspects, the histatin or analog thereof is the peptide represented by SEQ ID NO: 35.

In some aspects, the CNTF peptide is peptide 6, which is represented by SEQ ID NO: 34 in Table 1 below. In some aspects, the CNTF peptide is peptide 6c, which is represented by SEQ ID NO: 36.

In some aspects, the composition comprises the histatin or analog thereof at a concentration of from about 0.001 μM to about 100 mM. In some aspects, the composition comprises the histatin or analog thereof at a concentration of from about 0.001 μM to about 0.01 μM, from about 0.001 μM to about 0.1 μM, from about 0.001 μM to about 1 μM, from about 0.001 μM to about 10 μM, from about 0.001 μM to about 100 μM, from about 0.001 μM to about 1 mM, from about 0.001 μM to about 100 mM, from about 0.01 μM to about 0.1 μM, from about 0.01 μM to about 1 μM, from about 0.01 μM to about 10 μM, from about 0.01 μM to about 100 μM, from about 0.01 μM to about 1 mM, from about 0.01 μM to about 10 mM, from about 0.01 μM to about 100 mM, from about 0.1 μM to about 1 μM, from about 0.1 μM to about 10 M, from about 0.1 μM to about 100 μM, from about 0.1 μM to about 1 mM, from about 0.1 μM to about 10 mM, from about 0.1 μM to about 100 mM, from about 1 μM to about 10 μM, from about 1 μM to about 100 μM, from about 1 μM to about 1 mM, from about 1 μM to about 10 mM, from about 1 μM to about 100 mM, from about 10 μM to about 100 μM, from about 10 μM to about 1 mM, from about 10 μM to about 10 mM, from about 10 μM to about 100 mM, from about 100 μM to about 1 mM, from about 100 μM to about 10 mM, from about 100 μM to about 100 mM, from about 1 mM to about 10 mM, from about 1 mM to about 100 mM, or from about 10 mM to about 100 mM.

In some aspects, the composition comprises the histatin or analog thereof at a concentration of about 80 μM. In some aspects, the composition comprises the histatin or analog thereof at a concentration of about 0.001 μM, about 0.01 μM, about 0.1 μM, about 1 μM, about 10 μM, about 100 μM, about 1 mM, about 10 mM, or about 100 mM.

In some aspects, the composition comprises the CNTF peptide or analog thereof at a concentration of from about 0.001 μM to about 100 mM. In some aspects, the composition comprises the CNTF peptide or analog thereof at a concentration of from about 0.001 μM to about 0.01 μM, from about 0.001 μM to about 0.1 μM, from about 0.001 μM to about 1 μM, from about 0.001 μM to about 10 μM, from about 0.001 μM to about 100 μM, from about 0.001 μM to about 1 mM, from about 0.001 μM to about 100 mM, from about 0.01 μM to about 0.1 μM, from about 0.01 μM to about 1 μM, from about 0.01 μM to about 10 μM, from about 0.01 μM to about 100 M, from about 0.01 μM to about 1 mM, from about 0.01 μM to about 10 mM, from about 0.01 μM to about 100 mM, from about 0.1 μM to about 1 μM, from about 0.1 μM to about 10 μM, from about 0.1 μM to about 100 μM, from about 0.1 μM to about 1 mM, from about 0.1 μM to about 10 mM, from about 0.1 μM to about 100 mM, from about 1 μM to about 10 μM, from about 1 μM to about 100 μM, from about 1 μM to about 1 mM, from about 1 μM to about 10 mM, from about 1 μM to about 100 mM, from about 10 μM to about 100 μM, from about 10 μM to about 1 mM, from about 10 μM to about 10 mM, from about 10 μM to about 100 mM, from about 100 μM to about 1 mM, from about 100 μM to about 10 mM, from about 100 μM to about 100 mM, from about 1 mM to about 10 mM, from about 1 mM to about 100 mM, or from about 10 mM to about 100 mM.

In some aspects, the composition comprises the CNTF peptide or analog thereof at a concentration of about 100 μM. In some aspects, the composition comprises the CNTF peptide or analog thereof at a concentration of about 0.001 μM, about 0.01 μM, about 0.1 μM, about 1 μM, about 10 μM, about 50 μM, about 500 μM, about 1 mM, about 10 mM, or about 100 mM.

In some aspects, the composition comprises a therapeutically effective amount of an anti-apoptotic agent.

In some aspects, the composition comprises the anti-apoptotic agent at a concentration of from about 0.001 μM to about 100 mM. In some aspects, the composition comprises the anti-apoptotic agent at a concentration of from about 0.001 μM to about 0.01 μM, from about 0.001 μM to about 0.1 μM, from about 0.001 μM to about 1 μM, from about 0.001 μM to about 10 μM, from about 0.001 μM to about 100 μM, from about 0.001 μM to about 1 mM, from about 0.001 μM to about 100 mM, from about 0.01 μM to about 0.1 μM, from about 0.01 μM to about 1 μM, from about 0.01 μM to about 10 μM, from about 0.01 μM to about 100 μM, from about 0.01 μM to about 1 mM, from about 0.01 μM to about 10 mM, from about 0.01 μM to about 100 mM, from about 0.1 μM to about 1 μM, from about 0.1 μM to about 10 μM, from about 0.1 μM to about 100 μM, from about 0.1 μM to about 1 mM, from about 0.1 μM to about 10 mM, from about 0.1 μM to about 100 mM, from about 1 μM to about 10 μM, from about 1 μM to about 100 μM, from about 1 μM to about 1 mM, from about 1 μM to about 10 mM, from about 1 μM to about 100 mM, from about 10 μM to about 100 μM, from about 10 μM to about 1 mM, from about 10 μM to about 10 mM, from about 10 μM to about 100 mM, from about 100 μM to about 1 mM, from about 100 μM to about 10 mM, from about 100 μM to about 100 mM, from about 1 mM to about 10 mM, from about 1 mM to about 100 mM, or from about 10 mM to about 100 mM.

In some aspects, the composition comprises the anti-apoptotic agent at a concentration of about 80 μM. In some aspects, the composition comprises the anti-apoptotic agent at a concentration of about 0.001 μM, about 0.01 μM, about 0.1 μM, about 1 μM, about 10 μM, about 100 μM, about 1 mM, about 10 mM, or about 100 mM.

In some aspects, from about 1 μL to about 100 μL of the composition is administered to the subject. In some aspects, the composition is administered to one eye or both eyes of the subject. In some aspects, from about 1 μL to about 10 μL, from about 1 μL to about 100 μL, from about 1 μL to about 500 μL, from about 1 μL to about 1000 μL, from about 10 μL to about 100 μL, from about 10 μL to about 500 μL, from about 10 μL to about 1000 μL, from about 100 μL to about 500 μL, from about 100 μL to about 1000 μL, or from about 500 μL to about 1000 μL of the composition is administered to one eye or both eyes of the subject.

In some aspects, about 35 μL of the composition is administered to one eye or both eyes of the subject. In some aspects, the composition is administered to the eye or skin of the subject. In some aspects, about 1 μL, about 10 μL, about 50 μL, about 100 μL, about 500 μL, or about 1000 μL of the composition is administered to one eye or both eyes of the subject.

In some aspects, from about 0.1 mL to about 10 mL of the composition is administered to the subject. In some aspects, the composition is administered to the skin of the subject. In some aspects, from about 0.1 mL to about 0.5 mL, from about 0.1 mL to about 1 mL, from about 0.1 mL to about 5 mL, from about 0.5 mL to about 1 mL, from about 0.5 mL to about 5 mL, from about 0.5 mL to about 10 mL, from about 1 mL to about 5 mL, from about 1 mL to about 10 mL, or from about 5 mL to about 10 mL of the composition is administered to the skin of the subject.

In some aspects, about 1 mL of the composition is administered to the skin of the subject. In some aspects, about 0.1 mL, about 0.5 mL, about 5 mL, or about 10 mL of the composition is administered to the skin of the subject.

In some aspects, the composition is administered to the subject daily for from about 1 day to about 6 months. In some aspects, the composition is administered to the subject daily for from about 1 day to about 7 days, from about 1 day to about 14 days, from about 1 day to about 21 days, from about 1 day to about 1 month, from about 1 day to about 2 months, from about 1 day to about 3 months, from about 1 day to about 4 months, from about 1 day to about 5 months, from about 7 days to about 14 days, from about 7 days to about 21 days, from about 7 days to about 1 month, from about 7 days to about 2 months, from about 7 days to about 3 months, from about 7 days to about 4 months, from about 7 days to about 5 months, from about 7 days to about 6 months, from about 14 days to about 21 days, from about 14 days to about 1 month, from about 14 days to about 2 months, from about 14 days to about 3 months, from about 14 days to about 4 months, from about 14 days to about 5 months, from about 14 days to about 6 months, from about 21 days to about 1 month, from about 21 days to about 2 months, from about 21 days to about 3 months, from about 21 days to about 4 months, from about 21 days to about 5 months, from about 21 days to about 6 months, from about 1 month to about 2 months, from about 1 month to about 3 months, from about 1 month to about 4 months, from about 1 month to about 5 months, from about 1 month to about 6 months, from about 2 months to about 3 months, from about 2 months to about 4 months, from about 2 months to about 5 months, from about 2 months to about 6 months, from about 3 months to about 4 months, from about 3 months to about 5 months, from about 3 months to about 6 months, from about 4 months to about 5 months, from about 4 months to about 6 months, or from about 5 months to about 6 months.

In some aspects, the composition is administered to the subject daily for about 7 days. In some aspects, the composition is administered to the subject daily for about 1 day, about 14 days, about 21 days, about 1 month, about 2 months, about 3 months, about 4 months, about 5 months, or about 6 months.

In some aspects, the composition is administered to the subject three times per day. In some aspects, the composition is administered to the subject, one, two, four, five, six, seven, eight, nine, or ten times per day.

In some aspects, the composition is administered to the subject in 2 hour intervals. In some aspects, the composition is administered to the subject in 1 hour, 3 hour, 4 hour, 5 hour, or 6 hour intervals.

In some aspects, the second active agent is present in an amount known to be therapeutically (clinically) effective for the treatment of a target condition, disease or disorder, such as those described herein, when a unit dose of the pharmaceutical composition is administered to a subject in need thereof. In some aspects, the second active agent is a CNTF peptide or analog thereof. In other aspects, the second active agent is an anti-apoptotic agent. In some aspects, more than one additional active agent is present, such as both a CNTF peptide and an anti-apoptotic agent. If the active agents are present in such a weight ratio that a super-additive or synergistic therapeutic effect is observed upon administration to the subject, then the overall administered dose may be lowered, so that fewer undesired side-effects will occur.

In some aspects, the pharmaceutical composition comprises a combination of a histatin and one or more other active agents, and may contain an excess of a histatin, an excess of the one or more other active agents, or equivalent amounts of a histatin and the one or more other active agents. In some aspects, the one or more active agents is a CNTF peptide or analog thereof. In some aspects, the one or more active agents is an anti-apoptotic agent. The weight ratio of a histatin to the one or more other active agents can range from about 100:1 to 1:100. In some aspects, the one or more other active agents are administered to the subject sequentially or concomitantly in another dosage form such that the subject receives a dose of the histatin in the pharmaceutical composition and a dose of the one or more other active agents in another dosage form. The dosing regimen for the one or more other active agents can thus be the same as or different than the dosing regimen for the histatin. Sequential administration includes active agents administered within seconds, minutes, hours or days.

In some aspects, the weight ratio of a histatin to the one or more other active agents can range from about 80:1 to about 1:100, from about 60:1 to about 1:100, from about 40:1 to about 1:100, from about 20:1 to about 1:100, from about 10:1 to about 1:100, from about 5:1 to about 1:100, from about 2:1 to about 1:100, from about 1:1 to about 1:100, from about 1:2 to about 1:100, from about 1:5 to about 1:100, from about 1:10 to about 1:100, from about 1:20 to about 1:100, from about 1:40 to about 1:100, from about 1:60 to about 1:100, from about 1:80 to about 1:100, from about 100:1 to about 1:80, from about 80:1 to about 1:80, from about 60:1 to about 1:80, from about 40:1 to about 1:80, from about 20:1 to about 1:80, from about 10:1 to about 1:80, from about 5:1 to about 1:80, from about 2:1 to about 1:80, from about 1:1 to about 1:80, from about 1:2 to about 1:80, from about 1:5 to about 1:80, from about 1:10 to about 1:80, from about 1:20 to about 1:80, from about 1:40 to about 1:80, from about 1:60 to about 1:80, from about 100:1 to about 1:60, from about 80:1 to about 1:60, from about 60:1 to about 1:60, from about 40:1 to about 1:60, from about 20:1 to about 1:60, from about 10:1 to about 1:60, from about 5:1 to about 1:60, from about 2:1 to about 1:60, from about 1:1 to about 1:60, from about 1:2 to about 1:60, from about 1:5 to about 1:60, from about 1:10 to about 1:60, from about 1:20 to about 1:60, from about 1:40 to about 1:60, from about 100:1 to about 1:40, from about 80:1 to about 1:40, from about 60:1 to about 1:40, from about 40:1 to about 1:40, from about 20:1 to about 1:40, from about 10:1 to about 1:40, from about 5:1 to about 1:40, from about 2:1 to about 1:40, from about 1:1 to about 1:40, from about 1:2 to about 1:40, from about 1:5 to about 1:40, from about 1:10 to about 1:40, from about 1:20 to about 1:40, from about 100:1 to about 1:20, from about 80:1 to about 1:20, from about 60:1 to about 1:20, from about 40:1 to about 1:20, from about 20:1 to about 1:20, from about 10:1 to about 1:20, from about 5:1 to about 1:20, from about 2:1 to about 1:20, from about 1:1 to about 1:20, from about 1:2 to about 1:20, from about 1:5 to about 1:20, from about 1:10 to about 1:20, from about 100:1 to about 1:10, from about 80:1 to about 1:10, from about 60:1 to about 1:10, from about 40:1 to about 1:10, from about 20:1 to about 1:10, from about 10:1 to about 1:10, from about 5:1 to about 1:10, from about 2:1 to about 1:10, from about 1:1 to about 1:10, from about 1:2 to about 1:10, from about 1:5 to about 1:10, from about 100:1 to about 1:5, from about 80:1 to about 1:5, from about 60:1 to about 1:5, from about 40:1 to about 1:5, from about 20:1 to about 1:5, from about 10:1 to about 1:5, from about 5:1 to about 1:5, from about 2:1 to about 1:5, from about 1:1 to about 1:5, from about 1:2 to about 1:5, from about 100:1 to about 1:2, from about 80:1 to about 1:2, from about 60:1 to about 1:2, from about 40:1 to about 1:2, from about 20:1 to about 1:2, from about 10:1 to about 1:2, from about 5:1 to about 1:2, from about 2:1 to about 1:2, from about 1:1 to about 1:2, from about 100:1 to about 1:1, from about 80:1 to about 1:1, from about 60:1 to about 1:1, from about 40:1 to about 1:1, from about 20:1 to about 1:1, from about 10:1 to about 1:1, from about 5:1 to about 1:1, from about 2:1 to about 1:1, from about 100:1 to about 2:1, from about 80:1 to about 2:1, from about 60:1 to about 2:1, from about 40:1 to about 2:1, from about 20:1 to about 2:1, from about 10:1 to about 2:1, from about 5:1 to about 2:1, from about 100:1 to about 5:1, from about 80:1 to about 5:1, from about 60:1 to about 5:1, from about 40:1 to about 5:1, from about 20:1 to about 5:1, from about 10:1 to about 5:1, from about 5:1 to about 5:1, from about 100:1 to about 5:1, from about 80:1 to about 5:1, from about 60:1 to about 5:1, from about 40:1 to about 5:1, from about 20:1 to about 5:1, from about 10:1 to about 5:1, from about 100:1 to about 10:1, from about 80:1 to about 10:1, from about 60:1 to about 10:1, from about 40:1 to about 10:1, from about 20:1 to about 10:1, from about 100:1 to about 20:1, from about 80:1 to about 20:1, from about 60:1 to about 20:1, from about 40:1 to about 20:1, from about 100:1 to about 40:1, from about 80:1 to about 40:1, from about 60:1 to about 40:1, from about 100:1 to about 60:1, from about 80:1 to about 60:1, or from about 100:1 to about 80:1.

In some aspects, the weight ratio of a histatin to the one or more other active agents is about 1:1. In some aspects, the weight ratio of a histatin to the one or more other active agents is about 100:1, about 80:1, about 60:1, about 40:1, about 20:1, about 10:1, about 5:1, about 2:1, about 1:2, about 1:5, about 1:10, about 1:20, about 1:40, about 1:60, about 1:80, or about 1:100.

In some aspects, the pharmaceutical composition comprises a combination of a histatin analog or derivative, and one or more other active agents, and may contain an excess of a histatin analog or derivative, an excess of the one or more other active agents, or equivalent amounts of a histatin analog or derivative and the one or more other active agents. In some aspects, the one or more active agents is a CNTF peptide or analog thereof. In some aspects, the one or more active agents is an anti-apoptotic agent. The weight ratio of a histatin analog or derivative to the one or more other active agents can range from about 100:1 to 1:100. In some aspects, the one or more other active agents are administered to the subject sequentially or concomitantly in another dosage form such that the subject receives a dose of the histatin analog or derivative in the pharmaceutical composition and a dose of the one or more other active agents in another dosage form. The dosing regimen for the one or more other active agents can thus be the same as or different than the dosing regimen for the histatin analog or derivative. Sequential administration includes active agents administered within seconds, minutes, hours or days.

In some aspects, the composition is administered in the form of an eye drop, gel, ointment, or tissue glue, or by incorporating the composition into an ocular device or a contact lens worn by a subject.

In some aspects, the composition comprises one or more pharmaceutically acceptable excipients.

In some aspects, the composition may be formulated in any manner suitable for topical administration to an eye of a patient, i.e., as an ophthalmic composition. Numerous suitable topical ophthalmic drug forms are well-known (see, e.g. Baranowski et al., The Scientific World Journal, Vol. 2014, Article ID 861904, incorporated herein by reference). In various examples, the ophthalmic composition is in the form of a solution, a suspension, an emulsion, an ointment, a cream, a gel, a contact lens, an ocular insert, a depot, a matrix, an implantable, a drug delivery system, a pledget, a punctal plug, or a controlled or sustained release vehicle or system. In some aspects, the ophthalmic composition is in the form of a sustained release vehicle or system as described in U.S. Pat. Nos. 7,897,166, 6,991,802, 7,485,318, 10,149,825, 9,226,893, U.S. Pat. Publ. 2010/0119500, U.S. Pat. Nos. 9,949,922, 10,172,792, and 10,548,843, each of which is hereby incorporated by reference in its entirety. As used herein, “topical administration” includes administration of the ophthalmic composition to the surface of the cornea and/or to the surface of the conjunctiva. Topical administration also includes placement of the ophthalmic composition in the cul de sac of the eye. The ophthalmic composition may be aqueous or non-aqueous (e.g. mineral oil-based), but will generally be aqueous.

In some aspects, the ophthalmic formulation is incorporated into a biodegradable silica fiber spun from silica sol. In some aspects, the silica fiber has a solubility rate in simulated body fluid of from about 0.2 wt %/h to about 20 wt %/h.

In some aspects, the ophthalmic formulation is incorporated into a device made of a material suitable for medical use in humans and/or animals, said material bearing or being capable of binding a biologically active agent, wherein said material is multilayered and formed into a body of the shape of a finished device comprising
a) a core material, wherein said core material is formed into a body, optionally into a body having the shape of a finished device,
b) two or more layers of coating material of which the first layer has been applied onto said core material and additional layers have been applied onto said coating material of a preceding layer and
c) at least one layer of coating material capable of binding said biologically active agent.

In some aspects, the coating material is a biopolymer, a sol-gel produced silica gel, or a biologically active molecule. In some aspects, the layers of coating material asymmetric such that different layers cover different portions of the core material, different layers comprise different biologically active agents, or both.

In some aspects, the ophthalmic composition comprises a biodegradable carrier. In some aspects, the biodegradable carrier is a silica xerogel which is made from water and silane in a water: silane ratio range of from 35 to 75 by using an acid or a base as a catalyst. In some aspects, the silane is tetraethoxysilane. In some aspects, biodegradable carrier is in the form of monoliths, fibers, nets, cones, multiparticles or a coating suitable for an implant material.

In some aspects, the ophthalmic composition comprises bioresorbable siloxane prepared from tetraethoxysilane. In some aspects, the bioresorbable siloxane is a monolith. In some aspects, the bioresorbable siloxane is a coating. In some aspects, the bioresorbable siloxane is a particle.

In some aspects, the ophthalmic composition is in the form of a implant or delivery device comprising a water-soluble, undissolved silica-based material consisting of SiO₂ and/or hydrolysed SiO₂.

In some aspects, the composition is administered in any manner suitable for delivery of an active agent to the eye, generally referred to as “ophthalmic administration.” Ophthalmic administration to the eye encompasses, but is not limited to, intraocular injection, subretinal injection, intravitreal injection, periocular administration, subconjuctival injections, suprachoroidal injections, retrobulbar injections, intracameral injections (including into the anterior or vitreous chamber), sub-Tenon's injections or implants, ophthalmic solutions, ophthalmic suspensions, ophthalmic ointments, ocular implants and ocular inserts, intraocular solutions, use of iontophoresis, incorporation in surgical irrigating solutions, and packs (by way of example only, a saturated cotton pledget inserted in the formix).

In some aspects, the ophthalmic composition is a sterile liquid, such as a solution or suspension, contained within a multi-use or single-use eye drop dispensing bottle or vial. In a specific example, the ophthalmic composition is provided as a sterile liquid contained within a multi-use eye drop dispensing bottle or vial. Typically the eye drop formulation comprises an aqueous buffered saline solution such as phosphate or borate buffered saline from about pH 4 to about 8, typically from about pH 7.0 to about 8.0, and more typically from about pH 7.2 to about pH 7.4. The osmolality of the formulation is typically in the range of about 200, 250, or 270 mOsm/kg up to about 310, 350, or 400 mOsm/kg. A liquid formulation packaged in a multi-use eye drop dispensing bottle may contain an added preservative, such as benzalkonium chloride. However, in some examples, the ophthalmic composition may be preservative-free. As used herein, “preservative-free” means that the composition comprises no preservative agent in addition to the histatin or analog thereof, the CNTF peptide or analog thereof, and, optionally, an anti-apoptotic agent.

In some aspects, the composition, especially when formulated as a liquid, such as a solution or a suspension for dispensing by eye dropper, may contain an excipient that extends the period of time that a dose of the composition remains in contact with the cornea. For example, the excipient may be a viscosity-increasing agent and/or a mucoadhesive agent. Examples of such excipients include high molecular weight hydrophilic polymers including, but not limited to, polyvinyl alcohol, polyethylene glycol, carbomers, polycarbophil, polyoxyethlene-polyoxypropylene block copolymers (e.g. Poloxamer 407), cellulose derivatives (e.g. hydroxypropyl cellulose, hydroxypropyl methyl cellulose, carboxymethyl cellulose, and hydroxyethyl cellulose), natural polysaccharides (e.g. hyaluronic acid, dextran, chondroitin sulfate, gellan gum, xanthan gum, guar gum, trehalose, and tamarind seed polysaccharide). Additional mucoadhesive polymers are described in the literature (see e.g. Yadav et al. J. Chem. Pharm. Res., 2010, 2(5):418-432, incorporated herein by reference). Any combination of two or more of the foregoing polymers may be included in composition. As used herein, a high molecular weight polymer has a molecular weight of at least 100,000 daltons. In another example, the composition comprises a cyclodextrin (e.g. 2-hydroxypropyl-beta-cyclodextrin). In one example, the composition is formulated as a sustained release liquid. For example, the composition may be in the form of an ophthalmic suspension comprising mucoadhesive microspheres which sustain release of the histatin or analog thereof. The microspheres comprise a mucoadhesive polymer and a histatin or analog thereof. Methods of making mucoadhesive microspheres for ophthalmic suspensions are described in the literature (see e.g. Dandagi et al., Sci Pharm (2013) 81(1):259-280).

In some aspects, the composition comprises one or more viscosity agents. Non-limiting examples of viscosity agents include hydroxypropyl methylcellulose (HPMC), hydroxyethyl cellulose, hydroxypropyl cellulose, polyvinylpyrrolidone, carboxymethyl cellulose, polyvinyl alcohol, sodium chondroitin sulfate, and sodium hyaluronate. Other acceptable viscosity agents include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly (hydroxyethyl methacrylate), oxypolygelatin, pectin, polygeline, povidone, propylene carbonate, methyl vinyl ether/maleic anhydride copolymer (PVM/MA), poly (methoxyethyl methacrylate), poly (methoxyethoxyethyl methacrylate), silicon dioxide, polyvinylpyrrolidone (PVP: povidone), Splenda® (dextrose, maltodextrin and sucralose) and combinations thereof.

In some aspects, the composition comprises from about 0.1 wt % to about 1 wt % of one or more viscosity agents. In some aspects, the composition comprises from about 0.1 wt % to about 0.25 wt %, from about 0.1 wt % to about 0.5 wt %, from about 0.1 wt % to about 0.75 wt %, from about 0.25 wt % to about 0.5 wt %, from about 0.25 wt % to about 0.75 wt %, from about 0.25 wt % to about 1 wt %, from about 0.5 wt % to about 0.75 wt %, from about 0.5 wt % to about 1 wt %, or from about 0.75 wt % to about 1 wt % of one or more viscosity agents.

In some aspects, the viscosity of the composition is from about 10 cPs to about 30 cPs. In some aspects, the viscosity of the composition is from about 1 cPs to about 5 cPs, from about 1 cPs to about 10 cPs, from about 1 cPs to about 15 cPs, from about 1 cPs to about 20 cPs, from about 1 cPs to about 30 cPs, from about 1 cPs to about 40 cPs, from about 1 cPs to about 50 cPs, from about 1 cPs to about 60 cPs, from about 1 cPs to about 80 cPs, from about 1 cPs to about 100 cPs, from about 1 cPs to about 125 cPs, from about 1 cPs to about 150 cPs, from about 1 cPs to about 175 cPs, from about 1 cPs to about 200 cPs, from about 1 cPs to about 400 cPs, from about 5 cPs to about 10 cPs, from about 5 cPs to about 15 cPs, from about 5 cPs to about 20 cPs, from about 5 cPs to about 30 cPs, from about 5 cPs to about 40 cPs, from about 5 cPs to about 50 cPs, from about 5 cPs to about 60 cPs, from about 5 cPs to about 80 cPs, from about 5 cPs to about 100 cPs, from about 5 cPs to about 125 cPs, from about 5 cPs to about 150 cPs, from about 5 cPs to about 175 cPs, from about 5 cPs to about 200 cPs, from about 5 cPs to about 400 cPs, from about 10 cPs to about 15 cPs, from about 10 cPs to about 20 cPs, from about 10 cPs to about 30 cPs, from about 10 cPs to about 40 cPs, from about 10 cPs to about 50 cPs, from about 10 cPs to about 60 cPs, from about 10 cPs to about 80 cPs, from about 10 cPs to about 100 cPs, from about 10 cPs to about 125 cPs, from about 10 cPs to about 150 cPs, from about 10 cPs to about 175 cPs, from about 10 cPs to about 200 cPs, from about 10 cPs to about 400 cPs, from about 15 cPs to about 20 cPs, from about 15 cPs to about 30 cPs, from about 15 cPs to about 40 cPs, from about 15 cPs to about 50 cPs, from about 15 cPs to about 60 cPs, from about 15 cPs to about 80 cPs, from about 15 cPs to about 100 cPs, from about 15 cPs to about 125 cPs, from about 15 cPs to about 150 cPs, from about 15 cPs to about 175 cPs, from about 15 cPs to about 200 cPs, from about 15 cPs to about 400 cPs, from about 20 cPs to about 30 cPs, from about 20 cPs to about 40 cPs, from about 20 cPs to about 50 cPs, from about 20 cPs to about 60 cPs, from about 20 cPs to about 80 cPs, from about 20 cPs to about 100 cPs, from about 20 cPs to about 125 cPs, from about 20 cPs to about 150 cPs, from about 20 cPs to about 175 cPs, from about 20 cPs to about 200 cPs, from about 20 cPs to about 400 cPs, from about 30 cPs to about 40 cPs, from about 30 cPs to about 50 cPs, from about 30 cPs to about 60 cPs, from about 30 cPs to about 80 cPs, from about 30 cPs to about 100 cPs, from about 30 cPs to about 125 cPs, from about 30 cPs to about 150 cPs, from about 30 cPs to about 175 cPs, from about 30 cPs to about 200 cPs, from about 30 cPs to about 400 cPs, from about 40 cPs to about 50 cPs, from about 40 cPs to about 60 cPs, from about 40 cPs to about 80 cPs, from about 40 cPs to about 100 cPs, from about 40 cPs to about 125 cPs, from about 40 cPs to about 150 cPs, from about 40 cPs to about 175 cPs, from about 40 cPs to about 200 cPs, from about 40 cPs to about 400 cPs, from about 50 cPs to about 60 cPs, from about 50 cPs to about 80 cPs, from about 50 cPs to about 100 cPs, from about 50 cPs to about 125 cPs, from about 50 cPs to about 150 cPs, from about 50 cPs to about 175 cPs, from about 50 cPs to about 200 cPs, from about 50 cPs to about 400 cPs, from about 60 cPs to about 80 cPs, from about 60 cPs to about 100 cPs, from about 60 cPs to about 125 cPs, from about 60 cPs to about 150 cPs, from about 60 cPs to about 175 cPs, from about 60 cPs to about 200 cPs, from about 60 cPs to about 400 cPs, from about 80 cPs to about 100 cPs, from about 80 cPs to about 125 cPs, from about 80 cPs to about 150 cPs, from about 80 cPs to about 175 cPs, from about 80 cPs to about 200 cPs, from about 80 cPs to about 400 cPs, from about 100 cPs to about 125 cPs, from about 100 cPs to about 150 cPs, from about 100 cPs to about 175 cPs, from about 100 cPs to about 200 cPs, from about 100 cPs to about 400 cPs, from about 125 cPs to about 150 cPs, from about 125 cPs to about 175 cPs, from about 125 cPs to about 200 cPs, from about 125 cPs to about 400 cPs, from about 150 cPs to about 175 cPs, from about 150 cPs to about 200 cPs, from about 150 cPs to about 400 cPs, from about 175 cPs to about 200 cPs, from about 175 cPs to about 400 cPs, or from about 200 cPs to about 400 cPs.

In some aspects, the composition has a viscosity of about 30 cPs. In some aspects, the composition has a viscosity of about 1 cPs, about 5 cPs, about 10 cPs, about 15 cPs, about 20 cPs, about 30 cPs, about 40 cPs, about 50 cPs, about 60 cPs, about 80 cPs, about 100 cPs, about 125 cPs, about 150 cPs, about 175 cPs, about 200 cPs, or about 400 cPs.

In some aspects, the composition comprises about 0.1 wt % of one or more viscosity agents. In some aspects, the composition comprises about 0.25 wt %, about 0.5 wt %, about 0.75 wt %, or about 1 wt % of one or more viscosity agents.

In some aspects, the composition comprises one or more buffers. Non-limiting examples of buffers include acetate buffer, borate buffer, borate citrate buffer, citrate buffer, lactate buffer, phosphate buffer, succinate buffer, borate-polyol complex buffer, carbonate buffer, an organic buffer, an amino acid buffer, and combinations thereof.

Organic buffers include, but are not limited to, Good's Buffer, such as for example 2-(N-morpholino)ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4,-bis(2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; diethanolamine (DEA); and combinations thereof.

Amino acid buffers include, but are not limited to, taurine, aspartic acid and its salts (e.g., potassium salts, etc), ε-aminocaproic acid, and combinations thereof.

In some aspects, the pH of the ophthalmic formulation is adjusted by a strong acid or base. Examples of strong acids and strong bases are well known in the art and include, without limitation, NaOH, KOH, HCl, and H₂SO₄. In some aspects, the strong acid or base is HCl or NaOH.

In some aspects, the composition comprises from about 0.05 wt % to about 1 wt % of one or more buffers. In some aspects, the composition comprises from about 0.05 wt % to about 0.1 wt %, from about 0.05 wt % to about 0.25 wt %, from about 0.05 wt % to about 0.5 wt %, from about 0.05 wt % to about 0.75 wt %, from about 0.1 wt % to about 0.25 wt %, from about 0.1 wt % to about 0.5 wt %, from about 0.1 wt % to about 0.75 wt %, from about 0.1 wt % to about 1 wt %, from about 0.25 wt % to about 0.5 wt %, from about 0.25 wt % to about 0.75 wt %, from about 0.25 wt % to about 1 wt %, from about 0.5 wt % to about 0.75 wt %, from about 0.5 wt % to about 1 wt %, or from about 0.75 wt % to about 1 wt % of one or more buffers.

In some aspects, the composition comprises about 0.5 wt % of one or more buffers. In some aspects, the composition comprises about 0.05 wt %, about 0.1 wt %, about 0.25 wt %, about 0.75 wt %, or about 1 wt % of one or more buffers.

In some aspects, the composition has a pH of from about 7 to about 7.6. In some aspects, the composition has a pH of from about 6 to about 6.5, from about 6 to about 7, from about 6 to about 7.2, from about 6 to about 7.4, from about 6 to about 7.6, from about 6 to about 7.8, from about 6 to about 8, from about 6.5 to about 7, from about 6.5 to about 7.2, from about 6.5 to about 7.4, from about 6.5 to about 7.6, from about 6.5 to about 7.8, from about 6.5 to about 8, from about 7 to about 7.2, from about 7 to about 7.4, from about 7 to about 7.8, from about 7 to about 8, from about 7.2 to about 7.4, from about 7.2 to about 7.6, from about 7.2 to about 7.8, from about 7.2 to about 8, from about 7.4 to about 7.6, from about 7.4 to about 7.8, from about 7.4 to about 8, or from about 7.6 to about 7.8.

In some aspects, the composition has a pH of about 7.4. In some aspects, the composition has a pH of about 6, about 6.5, about 7, about 7.2, about 7.6, or about 8.

In some aspects, the composition comprises a preservative. Non-limiting examples of preservatives include benzalkonium chloride, stabilized oxychloro complexes (Purite®), phenylmercuric acetate, chlorobutanol, benzyl alcohol, parabens, and thimerosal. In some aspects, the composition comprises benzalkonium chloride.

In some aspects, the composition comprises from about 0.0075 wt % to about 0.0125 wt % benzalkonium chloride. In some aspects, the composition comprises from about 0.0025 wt % to about 0.005 wt %, from about 0.0025 wt % to about 0.0075 wt %, from about 0.0025 wt % to about 0.01 wt %, from about 0.0025 wt % to about 0.0125 wt %, from about 0.0025 wt % to about 0.02 wt %, from about 0.005 wt % to about 0.0075 wt %, from about 0.005 wt % to about 0.01 wt %, from about 0.005 wt % to about 0.0125 wt %, from about 0.005 wt % to about 0.02 wt %, from about 0.0075 wt % to about 0.01 wt %, from about 0.0075 wt % to about 0.02 wt %, from about 0.01 wt % to about 0.0125 wt %, from about 0.01 wt % to about 0.02 wt %, or from about 0.0125 wt % to about 0.02 wt % benzalkonium chloride.

In some aspects, the composition comprises about 0.01 wt % benzalkonium chloride. In some aspects, the composition comprises about 0.0025 wt %, about 0.005 wt %, about 0.0075 wt %, about 0.0125 wt %, or about 0.02 wt % benzalkonium chloride.

In some aspects, the composition does not contain a preservative.

In some aspects, the composition comprises one or more stabilizers. Stabilizers include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some aspects, amide analogues of stabilizers are also used. In some aspects, the chosen stabilizer changes the hydrophobicity of the composition, improves the mixing of various components in the composition, controls the moisture level in the composition, or controls the mobility of the phase.

In some aspects, the composition comprises one or more stabilizers in sufficient amounts to inhibit the degradation of the active agents. Examples of such stabilizing agents, include, but are not limited to: glycerol, methionine, monothioglycerol, EDTA, ascorbic acid, polysorbate 80, polysorbate 20, arginine, heparin, dextran sulfate, cyclodextrins, pentosan polysulfate and other heparinoids, divalent cations such as magnesium and zinc, or combinations thereof.

In some aspects, the solubility of the components of the composition may be enhanced by a surfactant or other appropriate co-solvent in the composition. Such co-solvents include polysorbate 20, 60, and 80, Pluronic F68, F-84 and P-103, cyclodextrin, or other agents known to those skilled in the art. In some aspects, the concentration of the co-solvent is from 0.01 wt % to about 2 wt %.

In some aspects, the composition comprises one or more polyols. As used herein, the term “polyol” includes any compound having at least one hydroxyl group on each of two adjacent carbon atoms that are not in trans configuration relative to each other. The polyol can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol, sorbitol and propylene glycol. It is contemplated that the polyol may be comprised of two or more different polyols.

In some aspects, the composition comprises one or more anti-aggregation additives. Anti-aggregation additives enhance stability of the composition by reducing the rate of protein aggregation. Anti-aggregation additives include, but are not limited to, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

In some aspects, the composition comprises one or more antioxidants. Antioxidants include, but are not limited to, ascorbic acid, methionine, sodium thiosulfate, sodium metabisulfite, and combinations thereof. Metal chelating agents, thiol-containing compounds, and other general stabilizing agents may be acceptable antioxidants.

In some aspects, the composition comprises one or more osmolality agents. Osmolality agents include, but are not limited to, salts, particularly sodium chloride or potassium chloride, organic compounds such as propylene glycol, mannitol, sorbitol, dextrose, and glycerin.

In some aspects, the composition has an osmolality of from about 260 to about 365 mOsm/kg. In some aspects, the composition has an osmolality of from about 285 mOsm/kg to about 295 mOsm/kg, from about 285 mOsm/kg to about 305 mOsm/kg, from about 285 mOsm/kg to about 315 mOsm/kg, from about 285 mOsm/kg to about 325 mOsm/kg, from about 285 mOsm/kg to about 335 mOsm/kg, from about 285 mOsm/kg to about 345 mOsm/kg, from about 285 mOsm/kg to about 355 mOsm/kg, from about 285 mOsm/kg to about 365 mOsm/kg, from about 295 mOsm/kg to about 305 mOsm/kg, from about 295 mOsm/kg to about 315 mOsm/kg, from about 295 mOsm/kg to about 325 mOsm/kg, from about 295 mOsm/kg to about 335 mOsm/kg, from about 295 mOsm/kg to about 345 mOsm/kg, from about 295 mOsm/kg to about 355 mOsm/kg, from about 295 mOsm/kg to about 365 mOsm/kg, from about 305 mOsm/kg to about 315 mOsm/kg, from about 305 mOsm/kg to about 325 mOsm/kg, from about 305 mOsm/kg to about 335 mOsm/kg, from about 305 mOsm/kg to about 345 mOsm/kg, from about 305 mOsm/kg to about 355 mOsm/kg, from about 305 mOsm/kg to about 365 mOsm/kg, from about 315 mOsm/kg to about 325 mOsm/kg, from about 315 mOsm/kg to about 335 mOsm/kg, from about 315 mOsm/kg to about 345 mOsm/kg, from about 315 mOsm/kg to about 355 mOsm/kg, from about 315 mOsm/kg to about 365 mOsm/kg, from about 325 mOsm/kg to about 335 mOsm/kg, from about 325 mOsm/kg to about 345 mOsm/kg, from about 325 mOsm/kg to about 355 mOsm/kg, from about 325 mOsm/kg to about 365 mOsm/kg, from about 335 mOsm/kg to about 345 mOsm/kg, from about 335 mOsm/kg to about 355 mOsm/kg, from about 335 mOsm/kg to about 365 mOsm/kg, from about 345 mOsm/kg to about 355 mOsm/kg, from about 345 mOsm/kg to about 365 mOsm/kg, from about 355 mOsm/kg to about 365 mOsm/kg, from about 260 mOsm/kg to about 265 mOsm/kg, from about 260 mOsm/kg to about 275 mOsm/kg, from about 260 mOsm/kg to about 285 mOsm/kg, from about 260 mOsm/kg to about 295 mOsm/kg, from about 260 mOsm/kg to about 305 mOsm/kg, from about 260 mOsm/kg to about 315 mOsm/kg, from about 260 mOsm/kg to about 325 mOsm/kg, from about 260 mOsm/kg to about 335 mOsm/kg, from about 260 mOsm/kg to about 345 mOsm/kg, from about 260 mOsm/kg to about 355 mOsm/kg, from about 260 mOsm/kg to about 365 mOsm/kg, from about 265 mOsm/kg to about 275 mOsm/kg, from about 265 mOsm/kg to about 285 mOsm/kg, from about 265 mOsm/kg to about 295 mOsm/kg, from about 265 mOsm/kg to about 305 mOsm/kg, from about 265 mOsm/kg to about 315 mOsm/kg, from about 265 mOsm/kg to about 325 mOsm/kg, from about 265 mOsm/kg to about 335 mOsm/kg, from about 265 mOsm/kg to about 345 mOsm/kg, from about 265 mOsm/kg to about 355 mOsm/kg, from about 265 mOsm/kg to about 365 mOsm/kg, from about 275 mOsm/kg to about 285 mOsm/kg, from about 275 mOsm/kg to about 295 mOsm/kg, from about 275 mOsm/kg to about 305 mOsm/kg, from about 275 mOsm/kg to about 315 mOsm/kg, from about 275 mOsm/kg to about 325 mOsm/kg, from about 275 mOsm/kg to about 335 mOsm/kg, from about 275 mOsm/kg to about 345 mOsm/kg, from about 275 mOsm/kg to about 355 mOsm/kg, from about 275 mOsm/kg to about 365 mOsm/kg.

In some aspects, the composition has an osmolality of about 260 mOsm/kg, about 265 mOsm/kg, about 275 mOsm/kg, about 285 mOsm/kg, about 295 mOsm/kg, about 305 mOsm/kg, about 315 mOsm/kg, about 325 mOsm/kg, about 335 mOsm/kg, about 345 mOsm/kg, about 355 mOsm/kg, about 365 mOsm/kg, about 370 mOsm/kg, or about 375 mOsm/kg.

In some aspects, the composition is isotonic. In some aspects, the composition is hypotonic. In some aspects, the composition is hypertonic.

In some aspects, the ophthalmic composition is formulated as unit dose ocular insert for placement in the eye's cul de sac. Methods of making ocular inserts are described in the literature (see e.g. U.S. Pat. Nos. 4,730,013, 7,749,970, and U.S. Pat. Publ. 2012/0215184, which are incorporated herein by reference). An ocular insert is a solid unit dosage form comprising of a biodegradable matrix containing the active agent, which in the case of the present ophthalmic compositions, is a histatin or analog thereof, a CNTF peptide or analog thereof, and, optionally, an anti-apoptotic agent. The matrix is typically made from a high molecular weight polymer or a combination of high molecular weight polymers, such as the aforementioned hydrophilic polymers and additional polymers disclosed in the aforementioned patent publications that describe ocular inserts. The ocular insert may additionally comprise a lubricant to enhance comfort. Upon placement in the eye, the ocular insert dissolves or erodes over a period of several hours to a day, and in some cases over several days.

In some aspects, the disclosure provides a method of treating an ocular infection in a patient, comprising topically administering an ophthalmic composition comprising an effective amount of a histatin or analog thereof and/or a CNTF peptide or analog thereof, and, optionally, an anti-apoptotic agent to the patient's eye. In various specific examples the patient is a human or a veterinary patient, and/or the ocular infection is bacterial conjunctivitis or bacterial keratitis, and/or the ocular infection is caused by MRSA.

In some aspects, the amount of histatin and/or CNTF peptide or analog thereof, and, optionally, anti-apoptotic agent in the ophthalmic composition is effective for the treatment or prophylaxis of the ocular surgery, injury, infection, disease or disorder. In a specific example, the amount of histatin and/or CNTF peptide or analog thereof, and, optionally anti-apoptotic agent in the ophthalmic composition is effective for the treatment of the ocular surgery, injury, infection, disease or disorder. In another example, the amount of histatin and/or CNTF peptide or analog thereof, and, optionally, anti-apoptotic agent in the ophthalmic composition is effective for the pretreatment or prophylaxis of the ocular surgery, injury, infection, disease or disorder. As used herein, “pretreatment or prophylaxis” means that ocular surgery, injury, infection, disease or disorder does not lead to keratocyte loss, or results in reduced keratocyte loss, when the eye is dosed with the ophthalmic composition (either before or after the ocular surgery, injury, infection, disease or disorder), whereas an eye not dosed with the ophthalmic composition (or treated with a control composition that lacks the histatin but is otherwise identical) results in statistically significant keratocyte loss.

Prophylaxis may be, for example, prophylaxis from infection or injury following surgery, for example photorefractive keratectomy surgery or other ocular surgery, injury, infection, disease, or disorder.

In some aspects, the composition comprising a histatin or analog thereof and/or a CNTF peptide or analog thereof, and, optionally, an anti-apoptotic agent is administered to a subject before the subject undergoes surgery. In some aspects, the surgery is photorefractive keratectomy (PRK) surgery. In some aspects, the surgery is penetrating keratoplasty (PK). In some aspects, the surgery is Descemet stripping automated endothelial keratoplasty (DSAEK) or Descemet's membrane endothelial keratoplasty (DMEK).

In some aspects, the composition comprising a histatin or analog thereof and/or a CNTF peptide or analog thereof, and, optionally, an anti-apoptotic agent is administered to a subject after the subject undergoes surgery. In some aspects, the surgery is photorefractive keratectomy (PRK) surgery. In some aspects, the surgery is penetrating keratoplasty (PK). In some aspects, the surgery is Descemet stripping automated endothelial keratoplasty (DSAEK) or Descemet's membrane endothelial keratoplasty (DMEK).

In some aspects, the subject is a mammal. In some aspects, the mammal is a rabbit, a horse, a dog, a cat, a cow, a pig, or a sheep. In some aspects, the mammal is a human.

In some aspects, administration of the composition results in a reduction in loss of keratocytes after an injury or surgery.

In some aspects, administration of the composition results in a restoration of keratocytes that have been lost after an injury or surgery.

In some aspects, loss of keratocytes in a subject is caused by photorefractive keratectomy, Fuch's corneal dystrophy, or keratoconus. In some aspects, loss of keratocytes in a subject is caused by photodamage to the skin. In some aspects, loss of keratocytes in a subject is caused by chemotherapy.

In one aspect, the histatin is a peptide including 8 to 44 amino acids. In some aspects, the peptide is an L-peptide. In other aspects, the peptide is a cyclic peptide.

In some aspects, the amino acid sequence of the histatin peptide is one or more of SEQ ID NOs: 1-33, and SEQ ID NOs: 38-40, or any combinations of these sequences. In some aspects, one or more of the amino acid sequences have a substitution, deletion and/or insertion of up to 3 amino acids. In some aspects, one or more of the amino acid sequences have a substitution, a deletion and/or an insertion of two or less amino acids. In other aspects, one or more of the amino acid sequences have a substitution, a deletion, and/or an insertion in one amino acid.

The SEQ ID NO: 4 peptide is also known as Histatin 1 (Hst-1). In some aspects, the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOs: 1-3, and 7-29 (for Histatin 1, Histatin 2 and Histatin 3) and SEQ ID NO: 32 (for Histatin 5) to facilitate wound closure in the disclosures described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used. Any histatin sequences could be cyclized and used.

In some aspects, a histatin described herein can include histatin 1-5. Examples of a histatin include, without limitation, a human histatin-1 having the amino acid sequence set forth in SEQ ID NO: 4. In some aspects, a histatin analog, derivative, fragment, or fusion is also contemplated. In some aspects, a histatin analog, derivative, fragment, or fusion of a histatin 1-5 as set forth in SEQ ID NOs: 1-33 or SEQ ID NOs: 38-40 is also contemplated. In some aspects, two or more histatin analogs, derivatives, fragments, or fusions of a histatin 1-5 as set forth in SEQ ID NOs: 1-33 or SEQ ID NOs: 38-40 connected by a linker is also contemplated. In some aspects, a histatin can have at least 70%, at least 75%, at least 80%, at least 82%, at least 85%, at least 88%, at least 90%, at least 91%, at least 92%, at least 93%, at least 94%, at least 95%, at least 96%, at least 97%, at least 98%, or at least 99% sequence identity to SEQ ID NOs: 1-33 or SEQ ID NOs: 38-40.

In some aspects, a ciliary neurotrophic factor (CNTF) peptide, peptide 6 described herein has the amino acid sequence set forth in SEQ ID NO: 34. In some aspects, a ciliary neurotrophic factor (CNTF) peptide, peptide 6 derivative is also contemplated. In some aspects, a CNTF peptide analog, derivative, fragment, or fusion of a CNTF peptide as set forth in SEQ ID NO: 34 is also contemplated. In some aspects, two or more CNTF peptides or analogs, derivatives, fragments, or fusions of a CNTF peptide as set forth in SEQ ID NO: 34 connected by a linker is also contemplated. In some aspects, a derivative of ciliary neurotrophic factor (CNTF) peptide, peptide 6, can have at least 70%, at least 75%, at least 80%, at least 82%, at least 85%, at least 88%, at least 90%, at least 91%, at least 92%, at least 93%, at least 94%, at least 95%, at least 96%, at least 97%, at least 98%, or at least 99% sequence identity to SEQ ID NO: 34.

The sequences of histatins and CNTF peptides are described in US 2013/0310327, WO 2021/108482, and US 2020/0108117, which are herein incorporated by reference in their entireties.

The disclosure provides sequences in Table 1 below:

| SEQ | |
|---|---|
| ID NO | Sequence |
| 1 | Ser Asn Tyr Leu Tyr Asp Asn |
| 2 | Arg Glu Phe Pro Phe Tyr Gly Asp Tyr |
| 3 | Glu Lys His His Ser His |
| 4 | Asp Ser His Glu Lys Arg His His Gly Tyr |
| | Arg Arg Lys Phe His Glu Lys His His Ser |
| | His Arg Glu Phe Pro Phe Tyr Gly Asp Tyr |
| | Gly Ser Asn Tyr Leu Tyr Asp Asn |
| 5 | Arg Lys Phe His Glu Lys His His Ser His |
| | Arg Glu Phe Pro Phe Tyr Gly Asp Tyr Gly |
| | Ser Asn Tyr Leu Tyr Asp Asn |
| 6 | Asp Ser His Ala Lys Arg His His Gly Tyr |
| | Lys Arg Lys Phe His Glu Lys His His Ser |
| | His Arg Gly Tyr Arg Ser Asn Tyr Leu Tyr |
| | Asp Asn |
| 7 | Glu Lys His His Ser His Arg Glu Phe Pro |
| | Phe Tyr Gly Asp Tyr |
| 8 | Arg Glu Phe Pro Phe Tyr Gly Asp Tyr Gly |
| | Ser Asn Tyr Leu Tyr Asp Asn |
| 9 | Ser His Arg Glu Phe Pro Phe Tyr Gly Asp |
| | Tyr Gly Ser Asn Tyr Leu Tyr Asp Asn |
| 10 | His His Ser His Arg Glu Phe Pro Phe Tyr |
| | Gly Asp Tyr Gly Ser Asn Tyr Leu Tyr Asp |
| | Asn |
| 11 | Glu Lys His His Ser His Arg Glu Phe Pro |
| | Phe Tyr Gly Asp Tyr Gly Ser Asn Tyr Leu |
| | Tyr Asp Asn |
| 12 | Phe His Glu Lys His His Ser His Arg Glu |
| | Phe Pro Phe Tyr Gly Asp Tyr Gly Ser Asn |
| | Tyr Leu Tyr Asp Asn |
| 13 | Arg Lys Phe His Glu Lys His His Ser His |
| | Arg Glu Phe Pro Phe Tyr Gly Asp Tyr |
| 14 | Glu Lys His His Ser His Arg Glu Phe Pro |
| | Phe Tyr Gly Asp Tyr |
| 15 | Arg Lys Phe His Glu Lys His His Ser His |
| | Arg Glu Phe Pro Phe Tyr Gly Asp Tyr Gly |
| | Ser |
| 16 | Glu Lys His His Ser His Arg Glu Phe Pro |
| | Phe Tyr Gly Asp Tyr Gly Ser |
| 17 | Arg Lys Phe His Glu Lys His His Ser His |
| | Arg Glu Phe Pro Phe Tyr Gly Asp Tyr Gly |
| | Ser Asn Tyr |
| 18 | Glu Lys His His Ser His Arg Glu Phe Pro |
| | Phe Tyr Gly Asp Tyr Gly Ser Asn Tyr |
| 19 | Arg Lys Phe His Glu Lys His His Ser His |
| | Arg Glu Phe Pro Phe Tyr Gly Asp Tyr Gly |
| | Ser Asn Tyr Leu Tyr |
| 20 | Glu Lys His His Ser His Arg Glu Phe Pro |
| | Phe Tyr Gly Asp Tyr Gly Ser Asn Tyr Leu |
| | Tyr |
| 21 | Arg Lys Phe His Glu Lys His His Ser His |
| | Arg Glu Phe Pro Phe Tyr Gly |
| 22 | Glu Lys His His Ser His Arg Glu Phe Pro |
| | Phe Tyr Gly |
| 23 | His His Ser His Arg Glu Phe Pro Phe Tyr |
| | Gly Asp Tyr Gly Ser Asn Tyr Leu Tyr |
| 24 | Ser His Arg Glu Phe Pro Phe Tyr Gly Asp |
| | Tyr Gly Ser Asn Tyr Leu Tyr |
| 25 | Ser His Arg Glu Phe Pro Phe Tyr Gly Asp |
| | Tyr Gly Ser Asn Tyr |
| 26 | Phe Pro Phe Tyr Gly Asp Tyr Gly Ser Asn |
| | Tyr Leu Tyr Asp Asn |
| 27 | Phe Tyr Gly Asp Tyr Gly Ser Asn Tyr Leu |
| | Tyr Asp Asn |
| 28 | His His Ser His Arg Glu Phe Pro Phe Tyr |
| | Gly Asp Tyr Gly Ser |
| 29 | Ser His Arg Glu Phe Pro Phe Tyr Gly Asp |
| | Tyr Gly Ser |
| 30 | Asp Ser His Ala Lys Arg His His Gly Tyr |
| | Lys Arg Lys Phe His Glu Lys His His Ser |
| | His Arg Gly Tyr |
| 31 | Gly Tyr Lys Arg Lys Phe His Glu Lys His |
| | His Ser His Arg |
| 32 | Ser His Arg Glu Phe Pro Phe Tyr Gly Asp |
| | Tyr |
| 33 | Gly Gly Asp Ser His Glu Lys Arg His His |
| | Gly Tyr Arg Arg Lys Phe His Glu Lys His |
| | His Ser His Arg Glu Phe Pro Phe Tyr Gly |
| | Asp Tyr Gly Ser Asn Tyr Leu Tyr Asp Asn |
| | Leu Pro Glu Thr |
| 34 | Val Gly Asp Gly Gly Leu Phe Glu Lys Lys |
| | Leu |
| 35 | Ser His X Gly Tyr, wherein X is R, K, H, |
| | D or E |
| 36 | Asp Gly Gly Leu |
| 37 | His His Ile Tyr Leu Gly Ala Val Asn Tyr |
| | Ile Tyr |
| 38 | Asp Gly Gly Leu-adamantylated Gly |
| 39 | His His Ile Tyr Leu Gly Ala Val Asn Tyr |
| | Ile Tyr-NH₂ |
| 40 | Lys Arg Leu Phe Lys Glu Phe Leu Phe Ser |
| | Leu Arg Lys Tyr |
| 41 | His His Ile Tyr Leu Gly Ala Thr Asn Tyr |
| | Ile Tyr |

In some aspects, the disclosure provides methods of treating or prophylactically treating dermal or ophthalmic diseases, disorders, or conditions comprising administering to a subject in need thereof a composition comprising a histatin or analog thereof and/or a CNTF peptide or analog thereof, and, optionally, an anti-apoptotic agent.

In some aspects, the dermal disease, disorder, or condition is post-CO₂ recovery, thermal burn, decubitus ulcer, autonomic dysreflexia in spinal cord injury, diabetic ischemic ulcer, ulcer prophylaxis, skin graft recovery, trauma resulting in skill loss, post-cryo/electrodesiccation recovery, or post-Mohs/skin cancer wound healing. In some aspects, the ophthalmic disease, disorder, or condition is post-photorefractive keratectomy (PRK) surgery, glaucoma surgery, infectious corneal ulcer, dye eye disease, post-collagen crosslinking (CSX) for keratoconus, neurotrophic keratitis, Fuch's corneal dystrophy, contact lens intolerance, dry eye disease, or keratoconus progression inhibition. In some aspects, the ophthalmic disease, disorder, or condition is posterior polymorphous corneal dystrophy (PPCD), aphakic or pseudophakic bullous keratopathy (ABK/PBK), endothelial dysfunction caused by penetrating or blunt trauma, congenital hereditary endothelial dystrophy (CHED), iridocorneal endothelial (ICE) syndrome, refractory glaucoma, previous failed corneal grafts, or herpes simplex virus endotheliitis.

In some aspects, the disclosure provides methods of treating, preserving, and/or storing a biological sample prior to a surgical procedure comprising contacting the biological sample with a composition comprising a histatin or analog thereof and/or a CNTF peptide or analog thereof, and, optionally, an anti-apoptotic agent. In some aspects, the biological sample is donor tissue. In some aspects, the donor tissue is corneal tissue. In some aspects, the donor tissue is suitable for transplantation into a mammalian subject. In some aspects, the donor tissue is suitable for transplantation into a human subject. In some aspects, the method further comprises storing the biological sample contacted with the composition at a temperature of from about −10° C. to about 25° C.

In some aspects, the biological sample remains viable for about 0.5 days, about 1 day, about 2 days, about 3 days, about 7 days, about 14 days, about 21 days, about 30 days, about 60 days, or about 90 days. In some aspects, the biological sample remains viable for from about 0.5 days to about 1 day, from about 0.5 days to about 2 days, from about 0.5 days to about 3 days, from about 0.5 days to about 7 days, from about 0.5 days to about 14 days, from about 0.5 days to about 21 days, from about 0.5 days to about 30 days, from about 0.5 days to about 60 days, from about 0.5 days to about 90 days, from about 1 day to about 2 days, from about 1 day to about 3 days, from about 1 day to about 7 days, from about 1 day to about 14 days, from about 1 day to about 21 days, from about 1 day to about 30 days, from about 1 day to about 60 days, from about 1 day to about 90 days, from about 2 days to about 3 days, from about 2 days to about 7 days, from about 2 days to about 14 days, from about 2 days to about 21 days, from about 2 days to about 30 days, from about 2 days to about 60 days, from about 2 days to about 90 days, from about 3 days to about 7 days, from about 3 days to about 14 days, from about 3 days to about 21 days, from about 3 days to about 30 days, from about 3 days to about 60 days, from about 3 days to about 90 days, from about 7 days to about 14 days, from about 7 days to about 21 days, from about 7 days to about 30 days, from about 7 days to about 60 days, from about 7 days to about 90 days, from about 14 days to about 21 days, from about 14 days to about 30 days, from about 14 days to about 60 days, from about 14 days to about 90 days, from about 21 days to about 30 days, from about 21 days to about 60 days, from about 21 days to about 90 days, from about 30 days to about 60 days, from about 30 days to about 90 days, and from about 60 days to about 90 days.

In some aspects, the surgical procedure is photorefractive keratectomy (PRK) surgery, Descemet stripping automated endothelial keratoplasty (DSAEK), or Descemet's membrane endothelial keratoplasty (DMEK).

In some aspects, the disclosure provides methods of reducing or preventing loss of corneal endothelial cells and/or restoring corneal endothelial cells comprising administering to the eye of a subject in need thereof a therapeutically effective amount of a composition comprising a histatin or analog thereof and, optionally, an anti-apoptotic agent.

In some aspects, the anti-apoptotic agent is MET12 (SEQ ID NO:37). In some aspects, the anti-apoptotic agent is a MET variant (SEQ ID NO:41). In some aspects, the anti-apoptotic agent can have at least 70%, at least 75%, at least 80%, at least 82%, at least 85%, at least 88%, at least 90%, at least 91%, at least 92%, at least 93%, at least 94%, at least 95%, at least 96%, at least 97%, at least 98%, or at least 99% sequence identity to SEQ ID NO: 37 or SEQ ID NO:41. The sequence of MET12 is described in U.S. Pat. No. 8,343,931, which is incorporated herein by reference in its entirety.

In some aspects, the composition is administered by topical administration, intraocular injection, subretinal injection, intravitreal injection, periocular administration, subconjuctival injection, suprachoroidal injections, retrobulbar injection, intracameral injection, or sub-Tenon's injection.

In some aspects, loss of corneal endothelial cells is caused by Fuch's endothelial corneal dystrophy, posterior polymorphous corneal dystrophy, aphakic or pseudophakic bullous keratopathy, endothelial dysfunction caused by penetrating or blunt trauma, congenital hereditary endothelial dystrophy, iridocorneal endothelial syndrome, refractory glaucoma, previous failed corneal grafts, surgical trauma, or herpes simplex virus endotheliitus.

In some aspects, loss of corneal endothelial cells is caused by Fuch's endothelial corneal dystrophy.

In some aspects, the composition is administered prior to a surgical procedure. In some aspects, the composition is administered during a surgical procedure. In some aspects, the composition is administered after a surgical procedure. In some aspects, the surgical procedure is penetrating keratoplasty (PK), Descemet stripping automated endothelial keratoplasty (DSAEK), or Descemet's membrane endothelial keratoplasty (DMEK).

In some aspects, administering the composition results in about 20% reduction in loss of corneal endothelial cells compared to no treatment. In some aspects, administering the composition results in about 1%, about 5%, about 10%, about 15%, about 30%, about 40%, about 50%, about 60%, about 70%, about 80%, about 90%, or about 100% reduction in loss of corneal endothelial cells compared to no treatment. In some aspects, administering the composition results in from about 1% to about 5%, from about 1% to about 10%, from about 1% to about 15%, from about 1% to about 20%, from about 1% to about 30%, from about 1% to about 40%, from about 1% to about 50%, from about 1% to about 60%, from about 1% to about 70%, from about 1% to about 80%, from about 1% to about 90%, from about 1% to about 100%, from about 5% to about 10%, from about 5% to about 15%, from about 5% to about 20%, from about 5% to about 30%, from about 5% to about 40%, from about 5% to about 50%, from about 5% to about 60%, from about 5% to about 70%, from about 5% to about 80%, from about 5% to about 90%, from about 5% to about 100%, from about 10% to about 15%, from about 10% to about 20%, from about 10% to about 30%, from about 10% to about 40%, from about 10% to about 50%, from about 10% to about 60%, from about 10% to about 70%, from about 10% to about 80%, from about 10% to about 90%, from about 10% to about 100%, from about 15% to about 20%, from about 15% to about 30%, from about 15% to about 40%, from about 15% to about 50%, from about 15% to about 60%, from about 15% to about 70%, from about 15% to about 80%, from about 15% to about 90%, from about 15% to about 100%, from about 20% to about 30%, from about 20% to about 40%, from about 20% to about 50%, from about 20% to about 60%, from about 20% to about 70%, from about 20% to about 80%, from about 20% to about 90%, from about 20% to about 100%, from about 30% to about 40%, from about 30% to about 50%, from about 30% to about 60%, from about 30% to about 70%, from about 30% to about 80%, from about 30% to about 90%, from about 30% to about 100%, from about 40% to about 50%, from about 40% to about 60%, from about 40% to about 70%, from about 40% to about 80%, from about 40% to about 90%, from about 40% to about 100%, from about 50% to about 60%, from about 50% to about 70%, from about 50% to about 80%, from about 50% to about 90%, from about 50% to about 100%, from about 60% to about 70%, from about 60% to about 80%, from about 60% to about 90%, from about 60% to about 100%, from about 70% to about 80%, from about 70% to about 90%, from about 70% to about 100%, from about 80% to about 90%, from about 80% to about 100%, or from about 90% to about 100% reduction in loss of corneal endothelial cells compared to no treatment. In some aspects, the reduction in loss of corneal endothelial cells is determined by measuring the corneal endothelial cell density using corneal tomography.

In some aspects, the disclosure provides methods of restoring, regenerating, or promoting regeneration of corneal nerves comprising administering to the eye of a subject in need thereof a therapeutically effective amount of a composition comprising a histatin or analog thereof, and/or a ciliary neurotrophic factor (CNTF) peptide or analog thereof, and, optionally, an anti-apoptotic agent.

In some aspects, the composition comprises a histatin or analog thereof.

In some aspects, the composition comprises a histatin or analog thereof and a ciliary neurotrophic factor (CNTF) peptide or analog thereof.

In some aspects, the composition comprises a histatin or analog thereof, a ciliary neurotrophic factor (CNTF) peptide or analog thereof, and an anti-apoptotic agent.

In some aspects, the composition comprises a histatin or analog thereof and an anti-apoptotic agent.

In some aspects, the composition comprises a ciliary neurotrophic factor (CNTF) peptide or analog thereof and an anti-apoptotic agent.

In some aspects, the composition comprises a ciliary neurotrophic factor (CNTF) peptide or analog thereof.

In some aspects, the composition is administered by topical administration, intraocular injection, subretinal injection, intravitreal injection, periocular administration, subconjuctival injection, suprachoroidal injections, retrobulbar injection, intracameral injection, or sub-Tenon's injection.

In some aspects, loss of corneal nerves is caused by neurotrophic keratitis, dry eye disease, herpetic keratitis, leprosy, diabetes, keratoconjunctivitis sicca, or keratoconus.

In some aspects, loss of corneal nerves is caused by a surgical procedure. In some aspects, the surgical procedure is penetrating keratoplasty (PK), Descemet stripping automated endothelial keratoplasty (DSAEK), Descemet's membrane endothelial keratoplasty (DMEK), photorefractive keratectomy (PRK), radial keratotomy, or laser-assisted in situ keratomileusis (LASIK).

In some aspects, the composition is administered prior to a surgical procedure. In some aspects, the composition is administered during a surgical procedure. In some aspects, the composition is administered after a surgical procedure.

In some aspects, administering the composition results in about 50% reduction in loss of corneal nerves compared to no treatment. In some aspects, administering the composition results in about 1%, about 5%, about 10%, about 15%, about 30%, about 40%, about 50%, about 60%, about 70%, about 80%, about 90%, or about 100% reduction in loss of corneal nerves compared to no treatment. In some aspects, administering the composition results in from about 1% to about 5%, from about 1% to about 10%, from about 1% to about 15%, from about 1% to about 20%, from about 1% to about 30%, from about 1% to about 40%, from about 1% to about 50%, from about 1% to about 60%, from about 1% to about 70%, from about 1% to about 80%, from about 1% to about 90%, from about 1% to about 100%, from about 5% to about 10%, from about 5% to about 15%, from about 5% to about 20%, from about 5% to about 30%, from about 5% to about 40%, from about 5% to about 50%, from about 5% to about 60%, from about 5% to about 70%, from about 5% to about 80%, from about 5% to about 90%, from about 5% to about 100%, from about 10% to about 15%, from about 10% to about 20%, from about 10% to about 30%, from about 10% to about 40%, from about 10% to about 50%, from about 10% to about 60%, from about 10% to about 70%, from about 10% to about 80%, from about 10% to about 90%, from about 10% to about 100%, from about 15% to about 20%, from about 15% to about 30%, from about 15% to about 40%, from about 15% to about 50%, from about 15% to about 60%, from about 15% to about 70%, from about 15% to about 80%, from about 15% to about 90%, from about 15% to about 100%, from about 20% to about 30%, from about 20% to about 40%, from about 20% to about 50%, from about 20% to about 60%, from about 20% to about 70%, from about 20% to about 80%, from about 20% to about 90%, from about 20% to about 100%, from about 30% to about 40%, from about 30% to about 50%, from about 30% to about 60%, from about 30% to about 70%, from about 30% to about 80%, from about 30% to about 90%, from about 30% to about 100%, from about 40% to about 50%, from about 40% to about 60%, from about 40% to about 70%, from about 40% to about 80%, from about 40% to about 90%, from about 40% to about 100%, from about 50% to about 60%, from about 50% to about 70%, from about 50% to about 80%, from about 50% to about 90%, from about 50% to about 100%, from about 60% to about 70%, from about 60% to about 80%, from about 60% to about 90%, from about 60% to about 100%, from about 70% to about 80%, from about 70% to about 90%, from about 70% to about 100%, from about 80% to about 90%, from about 80% to about 100%, or from about 90% to about 100% reduction in loss of corneal nerves compared to no treatment. In some aspects, the reduction in loss of corneal nerves is determined by measuring the change in area of a corneal lesion or wound.

The present disclosure also provides methods of treating or preventing aging of the skin, cellulites, dry skin, splits, or wounds, comprising administering to a subject in need thereof a composition comprising a histatin or analog thereof and/or a CNTF peptide or analog thereof, and, optionally, an anti-apoptotic agent. In some aspects, the wound is an internal wound, an oral wound, a skin wound, an external wound, an ulcer, and/or a decubitus wound. In some aspects, the wound comprises damage to the eye, for example damage to the conjunctiva.

The present disclosure also provides methods of treating or preventing oral ulcers, oral aphthous lesions, burning mouth syndrome, burning tongue, psoriasis, eczema, and hair loss, comprising administering to a subject in need thereof a composition comprising a histatin or analog thereof and/or a CNTF peptide or analog thereof, and, optionally, an anti-apoptotic agent.

These types of skin damages and wounds are known to the person skilled in the art. An internal wound is a wound present in the body, for example due to a surgical incision. An oral wound is a wound present in the oral cavity. A skin wound is a wound present in the skin. An external wound is to be understood as a wound that is visible and accessible from outside the body. An ulcer is a lesion on the surface of the skin or a mucous surface. A decubitus wound is a wound or ulceration caused by prolonged pressure on the skin and tissues when one stay in one position for a long period of time, such as lying in bed. The bony areas of the body are the most frequently affected sites, which become ischemic under sustained and constant pressure. Aging of the skin is the change in appearance of the skin due to time or exposure to the environment or the health status of an individual. Cellulites is the definition used in cosmetics relating to a wobbly or dimpled appearance of the skin.

In some aspects, the methods disclosed herein are used for the treatment of a skin wound. Skin wounds can be wounds in the epidermis or dermis of the skin. There are several types of wounds in which the skin or tissue may be in need of repair: abrasions, lacerations, incisions, punctures and avulsions and burns. Use of a composition disclosed herein can improve the general health status of the skin. In another aspect, the methods disclosed herein are used for the treatment of an oral wound. Oral wounds are wounds in any part of the oral cavity wherein the oral mucosa is damaged. In another aspect, the methods described herein are used for the treatment of an internal wound. Internal wounds are wounds wherein cell layers of endodermal or mesodermal origin are damaged. Examples are wounds in arteries or veins, peritoneum or pericardium.

The present disclosure also provides methods of treating ocular surface diseases comprising administering to a subject in need thereof a composition comprising a histatin or analog thereof and/or a CNTF peptide or analog thereof, and, optionally, an anti-apoptotic agent. The ocular surface diseases may include, but are not limited to, dry eyes, corneal ulcerations and erosions, inflammatory and infectious keratitis and conjunctivitis, surgical interventions, and trauma.

The following examples are illustrative and do not limit the scope of the claimed aspects.

### EXAMPLES

### Example 1

### Effect of Histatin-1 on Loss of Stromal Keratocytes

### Methods

### Animals

Twenty-four New Zealand White rabbits, approximately 4-6 months old and weighing 2-3 kg were divided into 3 groups of 8 animals and dosed topically three times per day (TID) for 28 days with either 35 μl Phosphate Buffered Saline (PBS), 35 μl 80 μM histatin-1 (Hst-1), or 35 μl 80 μM histatin-1+100 μM peptide 6 (P6) combination following unilateral surgical keratectomy procedure.

### Surgical Keratectomy

Animals were administered buprenorphine (0.01-0.05 mg/kg, SC) for pain control just prior to the procedure. A second dose of buprenorphine (0.01-0.05 mg/kg, SC) was administered in the evening, and then administered on an as needed basis. Animals was induced for surgery with a ketamine/xylazine cocktail (50 mg/kg ketamine and 10 mg/kg xylazine IM); anesthesia was maintained with isoflurane delivered in oxygen through a mask as needed. Following induction of anesthesia, 5% betadine solution, on autoclaved sterile gauze, was used to clean the periocular (eyelids) area of the surgical eye. Betadine was then used to irrigate the ocular surface and conjunctival cul-de-sac, which was then thoroughly irrigated with sterile eyewash. One drop each of 0.5% proparacaine HCl and 10% phenylephrine HCl, was then applied to the ocular surface for ocular anesthesia and mydriasis, respectively. The rabbit was positioned under an operating microscope and an eyelid speculum was affixed. An adjustable, 8 mm corneal vacuum trephine (Moria Surgical, Antony, France) was used to create a central corneal defect of approximately 10-25% stromal depth (approximately 100 μm). The trephined section of the cornea was removed, and the wound left open. Immediately following surgery, a single dose of topical antibiotic (Neomycin Polymyxin B Sulfates Gramicidin ophthalmic solution) was applied.

### Corneal Tomography

The corneas of anesthetized animals were imaged with a Heidelberg Retinal Tomograph fitted with a Rostock Corneal Module (HRT III RCM). This compact ophthalmic device uses confocal scanning laser microscopy to provide high-resolution en-face images of cornea cells and structures, including keratocytes subpopulations and the corneal sub-basal nerve plexus. Central cornea confocal scans from the epithelium to the endothelium were done and optical stacks of 400 μm×400 μm and 2 μm thick sections were generated and electronically stored for analysis.

### Sampling Protocol and Data Analysis

A sampling region of the stack of optical scans was selected that corresponded to approximately 150 μm above the endothelium. This was done by counting 75 images up from the endothelium and selecting 5 adjacent images to serve as replicates. Keratocytes presented as reflective distinct irregular shaped objects. Counts from the 5 images were then averaged and expressed as counts/field and displayed graphically as mean±standard deviation (SD). An unpaired Student's T test was used to compare the histatin-1 and the histatin-1+peptide 6 groups; p<0.05 was considered statistically significant.

### Results

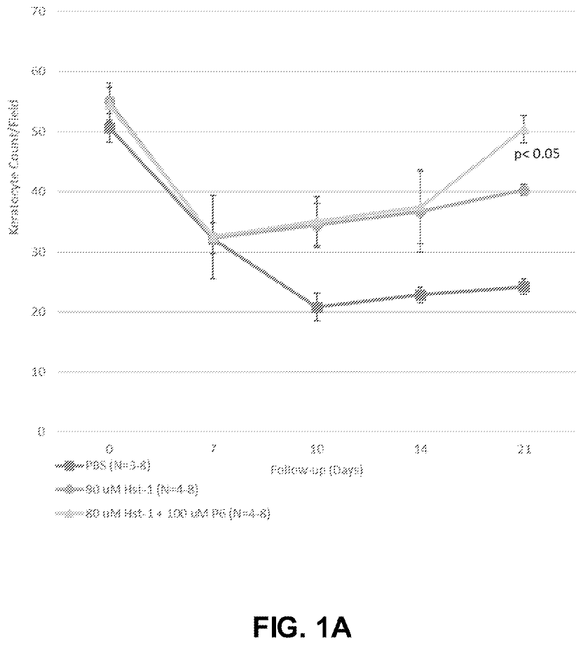

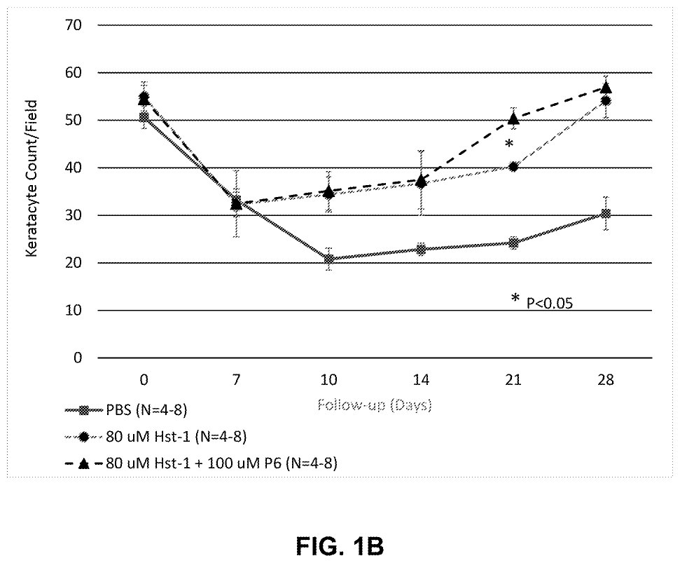

FIG. 1A shows the results of the experiment. Surgical keratectomy induced a gradual loss of stromal keratocytes which peaked at Day 10 and remained relatively unchanged through Day 21. Percent reductions from baseline (mean±SD) in the PBS group for Days 7, 10, 14 and 21 were 35.7±3, 58.6±5.5, 54.6±3.7 and 52±2.6, respectively. The response in the histatin-1 and histatin-1+peptide 6 combination groups at Day 7 were like the PBS group with percent keratocyte reductions of 40.2±3.3 and 42.4±14.2, respectively. Both drug groups showed significant reductions in the loss of keratocytes at Days 10, 14 and 21. Percent reduction for the histatin-1 only group for Days 10, 14 and 21 were 37.5±2.4, 32.7±15.3 and 26.5±6.0, respectively; for the combination group, the percent reductions were 32.7±9.5, 28.1±13.7 and 3.7±4.4. Interestingly, a comparison of Day 21 results between the histatin-1 and the histatin-1+peptide 6 combination groups show a statistical significantly (p=0.0009) and meaningful difference indicating that the inclusion of peptide 6 in the topical formulation appears to have increased the rate of keratocyte restoration.

FIG. 1B shows the results of the experiment through Day 28. Percent reductions from baseline (mean±SD) in the PBS group for Day 28 was 39.9±4.2. Both histatin-1 and histatin-1+peptide 6 combination groups showed significant reductions in the loss of keratocytes at Day 28. Percent reduction for the histatin-1 only group for Day 28 was 2.7±13; for the combination group, the percent reduction was −8.8±4.3. These data show complete recovery in the histatin-1 only group by Day 28 and in the combination group by Day 21 suggesting that the adjunctive use of peptide 6 provided for an improvement of the time to restoration by about 7 days.

### Example 2

### Effect of Histatin-1 on Corneal Endothelial Cell Density

### Methods

### Animals

Sixteen New Zealand White rabbits, approximately 4-6 months old and weighing 2-3 kg were divided into 2 groups of 8 animals and dosed topically three times per day (TID) for 28 days with either 35 μl phosphate buffered saline (PBS) or 35 μl 80 μM histatin-1 following unilateral surgical keratectomy procedure.

### Surgical Keratectomy

Animals were administered buprenorphine (0.01-0.05 mg/kg, SC) for pain control just prior to the procedure. A second dose of buprenorphine (0.01-0.05 mg/kg, SC) was administered in the evening, and then administered on an as needed basis. Animals were induced for surgery with a ketamine/xylazine cocktail (50 mg/kg ketamine and 10 mg/kg xylazine IM); anesthesia was maintained with isoflurane delivered in oxygen through a mask as needed. Following induction of anesthesia, 5% betadine solution, on autoclaved sterile gauze, was used to clean the periocular (eyelids) area of the surgical eye. Betadine was then used to irrigate the ocular surface and conjunctival cul-de-sac, which was then thoroughly irrigated with sterile eyewash. One drop each of 0.5% proparacaine HCl and 10% phenylephrine HCl, was then applied to the ocular surface for ocular anesthesia and mydriasis, respectively. The rabbits were positioned under an operating microscope and an eyelid speculum was affixed. An adjustable, 8 mm corneal vacuum trephine (Moria Surgical, Antony, France) was used to create a central corneal defect of approximately 10-25% stromal depth (approximately 100 μm). The trephined section of the cornea was removed, and the wound left open. Immediately following surgery, a single dose of topical antibiotic (Neomycin Polymyxin B Sulfates Gramicidin ophthalmic solution) was applied.

### Corneal Tomography

The corneas of anesthetized animals were imaged with a Heidelberg Retinal Tomograph fitted with a Rostock Corneal Module (HRT III RCM). This compact ophthalmic device uses confocal scanning laser microscopy to provide high-resolution en-face images of cornea cells and structures, including corneal sub-basal nerve plexus, keratocytes subpopulations and the corneal endothelial cell layer. Central cornea confocal scans from the epithelium to the endothelium were done and optical stacks of 400×400 μm and 2 μm thick sections were generated and electronically stored for analysis.

### Sampling Protocol and Data Analysis

The optical slice with the endothelium layer having the best resolution and largest coverage was selected. Of the 16 animals in the study, only 14 animals had scans with usable endothelial cell layers at 1 or more time points, and a maximum of only 4 usable scans at any single time point. The HRT III cell counting software was used to adjust image contrast and brightness to enhance manual cell counting within a selected region of interest (ROI). ROIs were >0.005 mm². Number of cells within a ROI ranged from 118-287. Partial cells (>½) at the edges of the ROI were counted only on 2 sides at right angles. The software computed endothelial cell density as cells/mm² based on manual counts in the selected ROI. Data are presented as mean±standard deviation cells/mm². An unpaired Student's T test was used to compare the PBS and histatin-1 groups at each follow-up time point; p<0.05 was considered statistically significant.

### Results

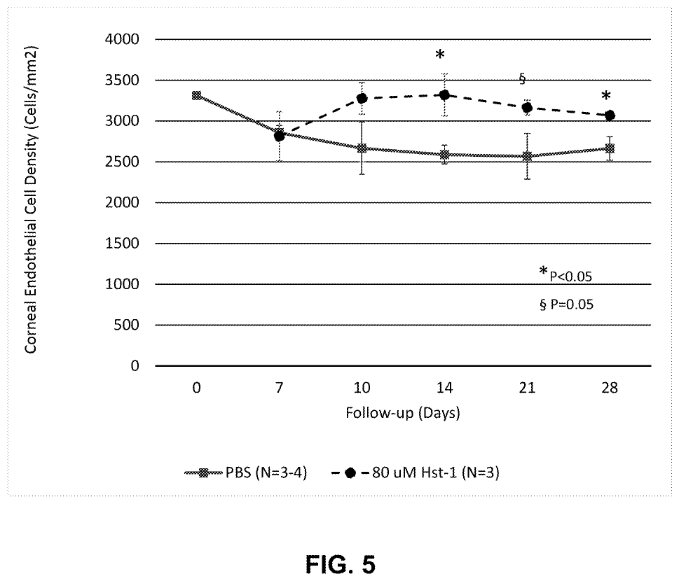

FIG. 5 shows the results of the experiment. Surgical anterior keratectomy induced a loss of endothelial cell density which peaked at Day 14 and remained depressed through Day 28. Percent reductions from baseline (mean±SD) in the PBS group for Days 7, 10, 14, 21 and 28 were 13.8±2.6, 19.5±9.6, 21.9±3.4, 22.5±8.4 and 20.2±5.0, respectively. The response in the histatin-1 group at Day 7 was like the PBS group with percent endothelial cell density reduction of 15.1±2.8 (p>0.05). Percent reduction for the histatin-1 group for Days 10, 14, 21 and 28 were 1.1±9.1, −0.2±5.9, 4.5±7.8 and 7.4±2.8, respectively. The histatin-1 response was significantly different from PBS (P<0.05) at Days 14, 21 and 28. These data show recovery and apparent normalization in the histatin-1 group starting by Day 10.

### Example 3

### Effect of Histatin-1 and Peptide 6 on Corneal Nerve Regeneration

### Methods

### Animals

Twelve New Zealand White rabbits, approximately 4-6 months old and weighing 2-3 kg were divided into 3 groups of 4 animals and dosed topically three times per day (TID) for 28 days with either 35 μl phosphate buffered saline (PBS), 35 μl 80 μM histatin-1 or 35 μl of 80 μM histatin-1+100 μM peptide 6 following a unilateral surgical keratectomy procedure.

### Surgical Anterior Keratectomy

Animals were administered buprenorphine (0.01-0.05 mg/kg, SC) for pain control just prior to the procedure. A second dose of buprenorphine (0.01-0.05 mg/kg, SC) was administered in the evening, and then administered on an as needed basis. Animals were induced for surgery with a ketamine/xylazine cocktail (50 mg/kg ketamine and 10 mg/kg xylazine IM); anesthesia was maintained with isoflurane delivered in oxygen through a mask as needed. Following induction of anesthesia, 5% betadine solution, on autoclaved sterile gauze, was used to clean the periocular (eyelids) area of the surgical eye. Betadine was then used to irrigate the ocular surface and conjunctival cul-de-sac, which was then thoroughly irrigated with sterile eyewash. One drop each of 0.5% proparacaine HCl and 10% phenylephrine HCl, was then applied to the ocular surface for ocular anesthesia and mydriasis, respectively. The rabbits were positioned under an operating microscope and an eyelid speculum was affixed. An adjustable, 8 mm corneal vacuum trephine (Moria Surgical, Antony, France) was used to create a central corneal defect of approximately 10-25% stromal depth (approximately 100 μm). The trephined section of the cornea was removed, and the wound left open. Immediately following surgery, a single dose of topical antibiotic (Neomycin Polymyxin B Sulfates Gramicidin ophthalmic solution) was applied.

### Corneal Wholemount Immunohistochemistry

### Eye Preparation

Eyes designated for immunohistochemistry were placed into Phosphate Buffered Saline (PBS) at room temperature (RT) and corneas excised with a 2-3 mm scleral margin using a #10 scalpel to create a small incision at the limbus and curved scissors to isolate each cornea. Using two pairs of forceps, the iris and lens were carefully peeled away and discarded. Descemet's Membrane (DM) was removed by making an incision of 0.5-1 mm near the intersection of the clear cornea/sclera with the scalpel. A reverse Terry-Sinskey hook was then used to scrape back and liberate a flap of DM. Corneas were then transferred to a dish containing PBS and the DM flap was grabbed and peeled back.

### Fixation

The cornea was fixed in 2% paraformaldehyde formulated in 0.1 M Phosphate Buffer, pH 7.4 for 2 h. Following fixation, corneas were incubated in 1/1,000 mouse anti-TUJ1 antibody (BioLegend cat #801213) and 1/300 goat anti-Ibal in 0.1 M PBS containing 1% normal donkey serum and 0.15% triton x-100 for 48 h at 4° C. Corneas were then washed 5×5 minutes in PBS and incubated with 1/500 donkey anti-mouse Cy2, 1/500 donkey anti-goat Cy3 and 1/5,000 DAPI in 0.1 M PBS containing 1% normal donkey serum and 0.15% triton x-100 for 15 h at 4° C. after which corneas were washed 5×5 minutes in PBS and mounted as described below.

### Slide Preparation

While corneas were incubating in their final wash, slides with imaging spacers were prepared. A 0.36, 0.6 mm or combination of the two adhesive imaging spacers (Grace Biolabs cat #SA-S-3L; custom cut to 22×40 mm rectangle with an oblong window cut out) was adhered to a glass slide; spacer thickness was determined by corneal thickness measured on the day of necropsy for treated eyes; control corneas utilized a 0.36 mm spacer.

### Mounting

After final wash, curved scissors were used to remove nearly all of the sclera margin. Then, using a #10 scalpel blade, 4-6 small incisions were made around the periphery to flatten the cornea. The cornea was then carefully placed within the spacer window, endothelial side facing down, and 3-4 drops of mounting media (90% glycerol, 0.5% N-propyl gallate, 20 mM Tris pH 8) applied using a 2 mL transfer pipette. The slide was then gently cover-slipped, taking care to avoid bubbles. Slides were imaged on an Olympus Bx63 upright fluorescent microscope using a 4× Fluorite objectives and images acquired using Olympus CellSens software. The central cornea region was imaged by tiling to create a composite of the entire surgical area

### Data Analysis

Greyscale Tuj1 images from Olympus CellSens software were imported into Fuji ImageJ software (NIH). The system was calibrated to the mm scale on the image. An 8 mm circle was then made and centered on the image to capture the area with the initial injury. The area external to 8 mm circle was removed. The image was sharpened to better visualize the nerves and divided into quadrants for ease of analysis. The Freehand selection tool was then used to map areas in the injured area that contained regenerated nerves. The measure tool was used to readout the area in mm². Data are expressed as percent of initial 8 mm lesion area of 50.3 mm² and presented as mean±standard deviation.

### Results

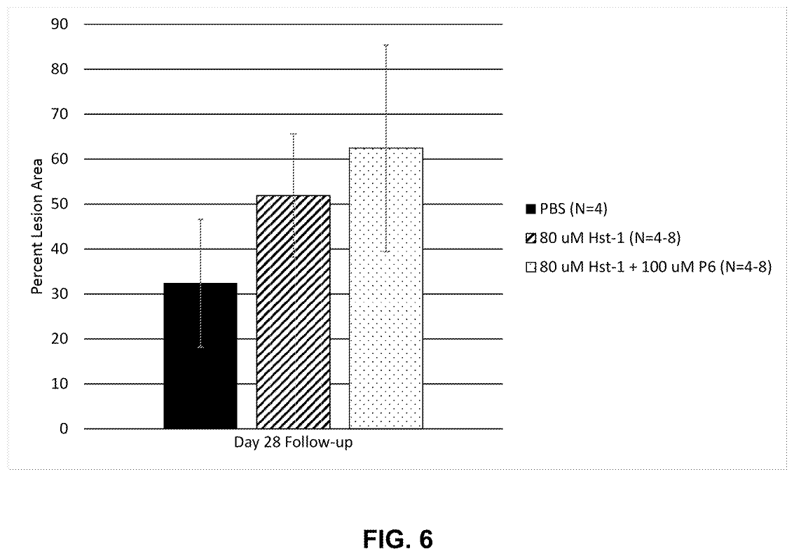

FIG. 6 shows the results of the experiment. Surgical keratectomy resulted in a reduction of ˜70% of area covered by corneal nerves at Day 28. The area of nerve regeneration in animals treated with PBS was 16.3±7 mm² which is 32.4±14% of initial lesion area. The area of nerve regeneration in eyes treated with histatin-1 was 26.1±7 mm² which is equivalent to 51.9±14% of lesion area. The adjunctive use of peptide 6 produced the strongest effect: area of nerve regeneration was 31.4±12 mm² which is equivalent to 63.4±11% of lesion area. These data show better nerve regeneration in the lesion area with the following ranked order of potency: histatin-1+peptide 6>histatin-1.

### Example 4

### Effect of Histatin-1 on Corneal Button Preservation

### Methods

The effect of 1 μM histatin-1 formulated in Life4° C.™ or Optisol™ Corneal Storage Medium was assessed on the preservation of endothelial cells in 9 pairs of human non-surgical cornea buttons stored under stressed conditions. The tissues were individually stored in 20 mL Bausch & Lomb™ Cornea Tissue Viewing Chambers at room temperature (24±2° C.) for 6 to 14 days, preceded by storage at 4° C. for 3 days. All doners were White/Caucasian origin. The median age was 64 years and ranged from 14 to 73. The amount of time from the time of death to the preservation of the corneas ranged from 4 hours 15 mins to 20 hours 48 mins. Endothelial cell counts at the time of tissue harvest was 2222±240 cells/mm². Histatin-1 was added every other day to the right eye (OD) in a volume of 200 μL in an amount sufficient to maintain a concentration of 1 μM. Left eyes (OS) were given an equal volume of PBS. Endothelial cell viability was assessed by trypan blue staining as non-viable area expressed as a percentage of 80% of the central cornea. Briefly, cornea buttons were transferred from storage chambers to viewing wells (Barron Punch Cutting Block), endothelium side up, and 0.06% trypan blue in a volume sufficient to cover the endothelial layer limbus to limbus was applied and left for 90 secs, and then rinsed twice with PBS. Uptake of the trypan blue stain indicated epithelial cell death, so the lesser extent of the staining is correlated with more viable tissue. Corneas was then imaged with a Zeiss Surgical Scope, model #STMI 508 equipped with a Zeiss eye piece camera Model #AXIOCCM 208. Image J (NIH) with the “Weka Segmentation” software was used to analyze the data.

### Results

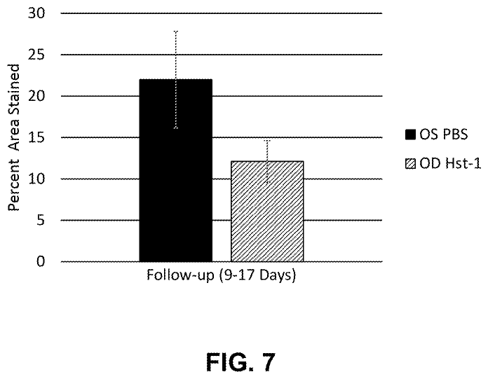

Area stained in the PBS and Histatin-1 groups were 22.0±5.8% and 12.1±2.9%, respectively; mean±SEM (see FIG. 7). Thus, the addition of Histatin-1 to the corneal buttons resulted in a 45% reduction of stained area in the endothelial area. These data suggest that human cornea buttons incubated with 1 μM Histatin-1 in standard cornea storage media under stressed conditions such as at room temperature will result in the significant preservation of endothelial cells loss and thus the enhanced preservation of cornea tissue in storage.

1. A method of reducing or preventing loss of keratocytes, keratinocytes, and/or keratoblasts, restoring keratocytes, keratinocytes, and/or keratoblasts, inhibiting apoptosis to prevent loss of cells in the eye or skin caused by injury or disease, preventing or minimizing refractive regression and subepithelial scarring, mobilizing keratocytes, keratinocytes, and/or keratoblasts, or treating corneal wounds, comprising administering to the eye or skin of a subject in need thereof a therapeutically effective amount of a composition comprising a histatin or analog thereof, and/or a ciliary neurotrophic factor (CNTF) peptide or analog thereof, and, optionally, an anti-apoptotic agent.

2. The method of claim 1, wherein the histatin or analog thereof is histatin-1 (SEQ ID NO:4).

3. The method of claim 1, wherein the ciliary neurotrophic factor (CNTF) peptide is peptide 6 (SEQ ID NO: 34).

4-13. (canceled)

14. The method of claim 1, wherein the composition is administered as an eye drop, gel, ointment, or tissue glue.

15. The method of claim 1, wherein the composition is administered in an ocular insert or contact lens.

16-17. (canceled)

18. The method of claim 1, wherein loss of keratocytes, keratinocytes, and/or keratoblasts is caused by ocular infection, corneal injury, ocular surgery, ocular injury, ocular disease, or ocular disorder.

19-21. (canceled)

22. The method of claim 1, wherein loss of keratocytes, keratinocytes, and/or keratoblasts is caused by skin photodamage.

23. The method of claim 1, wherein loss of keratocytes, keratinocytes, and/or keratoblasts is caused by chemotherapy.

24-43. (canceled)

44. A method of treating or prophylactically treating a dermal or ophthalmic disease, disorder, or condition, comprising administering to the eye or skin of a subject in need thereof a therapeutically effective amount of a composition comprising a histatin or analog thereof and/or a ciliary neurotrophic factor (CNTF) peptide or analog thereof.

45. The method of claim 44, wherein the composition further comprises an anti-apoptotic agent.

46. The method of claim 44, wherein the dermal disease, disorder, or condition is post-CO₂ recovery, thermal burn, decubitus ulcer, autonomic dysreflexia in spinal cord injury, diabetic ischemic ulcer, ulcer prophylaxis, skin graft recovery, trauma resulting in skill loss, post-cryo/electrodesiccation recovery, or post-Mohs/skin cancer wound healing.

47. The method of claim 44, wherein the ophthalmic disease, disorder, or condition is post-photorefractive keratectomy (PRK) surgery, glaucoma surgery, infectious corneal ulcer, dye eye disease, post-collagen crosslinking (CSX) for keratoconus, neurotrophic keratitis, Fuch's corneal dystrophy, contact lens intolerance, dry eye disease, keratoconus progression inhibition, posterior polymorphous corneal dystrophy (PPCD), aphakic or pseudophakic bullous keratopathy (ABK/PBK), endothelial dysfunction caused by penetrating or blunt trauma, congenital hereditary endothelial dystrophy (CHED), iridocorneal endothelial (ICE) syndrome, refractory glaucoma, previous failed corneal grafts, or herpes simplex virus endotheliitis.

48. The method of claim 47, wherein the ophthalmic disease, disorder, or condition is post-photorefractive keratectomy surgery, glaucoma surgery, or Fuch's corneal dystrophy.

49. The method of claim 44, wherein the histatin or analog thereof is histatin-1 (SEQ ID NO:4).

50. The method of claim 44, wherein the ciliary neurotrophic factor (CNTF) peptide or analog thereof is peptide 6 (SEQ ID NO: 34).

51-64. (canceled)

65. A method of storing a biological sample prior to a surgical procedure, comprising contacting the biological sample with a composition comprising a histatin or analog thereof and/or a CNTF peptide or analog thereof, and, optionally, an anti-apoptotic agent and storing said biological sample at a temperature of from about −10° C. to about 25° C.

66. The method of claim 65, wherein the biological sample is donor tissue.

67. The method of claim 66, wherein the donor tissue is corneal tissue.

68. The method of claim 65, wherein the biological sample remains viable for about 0.5 days to about 90 days.

69. The method of claim 65, wherein the surgical procedure is photorefractive keratectomy (PRK) surgery, Descemet stripping automated endothelial keratoplasty (DSAEK), or Descemet's membrane endothelial keratoplasty (DMEK).

70-117. (canceled)
